(19) United States
(12) Patent Application Publication
(10) Pub. No.: US 2023/0226123 A1
(43) Pub. Date: Jul. 20, 2023

(54) USE OF BACTERIA IN CHILDREN DEVELOPMENT ASSESSMENT AND TREATMENT

(71) Applicant: The Chinese University of Hong Kong, Shatin, New Territories (CN)

(72) Inventors: Siew Chien NG, Shatin, New Territories (CN); Ka Leung Francis CHAN, Tai Po, New Territories (CN); Zhilu XU, Shijiazhuang (CN); Yating WAN, Shen Zhen (CN)

(21) Appl. No.: 18/010,176

(22) PCT Filed: Jun. 15, 2021
(86) PCT No.: PCT/CN2021/099932

§ 371 Date: Dec. 13, 2022

Related U.S. Application Data
(60) Provisional application No. 63/121,198, filed on Dec. 3, 2020.
(60) Provisional application No. 63/039,034, filed on Jun. 15, 2020.

(51) Int. Cl. A61K 35/74; A61K 9/00; C12Q 1/689; A61P 25/28

(52) U.S. Cl. CPC A61K 35/74; A61K 9/0053; C12Q 1/689; A61P 25/28

(57) ABSTRACT

Provided are methods for treating symptoms of autism spectrum disorder (ASD) in a human child. Methods for determining risk for A SD in human children, methods for assessing developmental age of children and for treating children in need thereof, kits and compositions for use in these methods are also provided.

### RELATED APPLICATIONS

This application claims priority to U.S. Provisional Patent Application No. 63/039,034, filed Jun. 15, 2020, and U.S. Provisional Patent Application No. 63/121,198, filed, Dec. 3, 2020, the contents of all of the above are hereby incorporated by reference in the entirety for all purposes.

### BACKGROUND OF THE INVENTION

Autism spectrum disorder (ASD) is a complex group of developmental disorders characterized by impaired social interactions and communication together with repetitive behaviors. The purpose of this study is to determine bacterial biomarkers for individuals with autism, as well as to identify probiotic/therapeutic bacteria for autism. The gut bacterial profile is different between autism children and typically developing children, and the profile also evolves as children grow and develop. Gut microbiota is regarded as an important factor in the development of ASD as well as indicator of growth and development age of children. Currently there are no effective methods for diagnosing or treating autism and particularly no existing model using microbial markers to predict risk of autism in children, nor any model using microbial markers for assessing developmental age of children. This invention provide a new method for predicting risk of autism in children, new method for improve behavioral symptom in autism patients by microbial transfer and/or supplementation, and new method for assessing children's developmental age based on their gut microbial profile.

### BRIEF SUMMARY OF THE INVENTION

The invention relates to novel methods and compositions useful for treating the symptoms of autism spectrum disorder (ASD). In particular, the present inventor discovered that certain microorganism species, especially certain bacteria, are at an altered level, in the gastrointestinal (GI) tract of children at risk for ASD or suffering from ASD. Health benefits such as improving behavioral symptoms and alleviating detrimental effects can be achieved by modulating the level of pertinent microorganisms in patients' gut, for example, by fecal microbiota transplantation (FMT) treatment or oral administration of beneficial bacterial species or by suppressing the level of harmful bacterial species. These findings also provide new methods indicating the presence or risk of ASD in a child. Thus, in the first aspect, the present invention provides a novel method for treating ASD, including alleviating ASD symptoms, by increasing the level of one or more bacterial species named in Table 1 in the gastrointestinal tract of a child afflicted by ASD.

In some embodiments, the introducing step comprises oral administration to the subject a composition comprising an effective amount of the one or more of the bacterial species. In some embodiments, the introducing step comprises delivery to the small intestine, ileum, or large intestine of the subject a composition comprising an effective amount of the one or more of the bacterial species. In some embodiments, the introducing step comprises fecal microbiota transplantation (FMT). In some embodiments, the FMT comprises administration to the child a composition comprising processed donor fecal material. In some embodiments, the composition is orally administered; or the composition is directly deposited to the child's gastrointestinal tract. In some embodiments, the level or relative abundance of the one or more of the bacterial species is determined in a first stool sample obtained from the child prior to the introducing step and in a second stool sample obtained from the child after the introducing step. In some embodiments, the level of the one or more of the bacterial species is determined by polymerase chain reaction (PCR), especially quantitative PCR.

In a second aspect, the present invention provides a method for treating ASD, including alleviating ASD symptoms, by reducing the level of one or more bacterial species in Table 2 in the gastrointestinal tract of a child afflicted by ASD.

In some embodiments, the reducing step comprises FMT. In some embodiments, the reducing step comprises treating the subject with an anti-bacterial agent. In some embodiments, a composition comprising processed donor fecal material is introduced to the gastrointestinal tract of the subject after the subject is treated with the anti-bacterial agent. For example, the composition is orally administered, or the composition is directly deposited to the gastrointestinal tract of the child. In some embodiments, the level or relative abundance of the one or more of the bacterial species is determined in a first stool sample obtained from the child prior to the reducing step and in a second stool sample obtained from the child after the reducing step. In some embodiments, the level of the one or more bacterial species is determined by PCR, especially by quantitative PCR.

In a related aspect, a kit is provided for treating the symptoms of ASD. The kit comprises: a first container containing a first composition comprising (i) an effective amount of a first one of the bacterial species set forth in Table 1, or (ii) an effective amount of an anti-bacterial agent that suppresses growth of a first one of the bacterial species set forth in Table 2, and a second container containing a second composition comprising (i) an effective amount of a second one of the bacterial species set forth in Table 1, or (ii) an effective amount of an anti-bacterial agent that suppresses growth of a second one of the bacterial species set forth in Table 2.

In some embodiments, the first composition comprises processed donor fecal material for FMT, for example, the material has been processed and formulated for oral administration, such as dried, frozen or lyophilized, and placed in a capsule suitable for oral ingestion. In some embodiments, the second composition is formulated for oral administration. In some embodiments, both the first and second compositions are formulated for oral administration. In some cases, the kit may include two or more compositions each comprising an effective amount of at least one, possibly two or more, of the bacterial species set forth in Table 1, and/or (ii) an effective amount of an anti-bacterial agent that suppresses growth of at least one, possibly two or more, of the bacterial species set forth in Table 2. The compositions in the kit may each comprise a physiologically acceptable carrier or excipient.

In a third aspect, a method is provided for determining risk for autism spectrum disorder (ASD) in a human child. The method comprises these steps: (1) determining, in a stool sample taken from the child, the relative abundance of any one of the bacterial species set forth in Table 1 or 2; and (2) detecting the relative abundance from step (1) being no lower than the cutoff value in Table 1 or a standard control value or being lower than the cutoff value in Table 2 or a standard control value and determining the child as not having increased risk for ASD; or detecting the relative abundance from step (1) being lower than the cutoff value in Table 1 or a standard control value or being no lower than the cutoff value in Table 2 or a standard control value and determining the child as having an increased risk for ASD. In some embodiments, the relative abundance of the bacterial species in the child's stool sample is determined by PCR, e.g., quantitative PCR.

In a related aspect, a method is provided for assessing risk for autism spectrum disorder (ASD) in two human children. The method comprises these steps: (1) determining, in a stool sample from each of the two children, the relative abundance of any one of the bacterial species set forth in Table 1 or 2; and (2) determining the relative abundance of a bacterial species set forth in Table 1 from step (1) being higher in the stool sample from the first child or the relative abundance of a bacterial species set forth in Table 2 from step (1) being lower in the stool sample from the first child; and (3) determining the second child as having a higher risk for ASD than the first child. In some embodiments, the relative abundance of the bacterial species in both children's stool samples is determined by PCR, e.g., quantitative PCR.

Further, a method is provided for determining risk for ASD in a human child including these steps: (1) determining in a stool sample from the child a value of (a) the relative abundance of Alistipes indistinctus (Ai) or Anaerotruncus colihominis (Ac), or (b) the combined score of levels of three bacterial species Ai, Ac, and Eubacterium hallii (Eh), which is calculated by I1+β1*Ai+β2*Eh+β3*Ac; and (2) detecting the value to be higher than a standard control value and determining the individual as having increased risk of ASD.

Similarly, a method is provided for determining risk of ASD in a human child including these steps: (1) determining in a stool sample from the child relative abundance of Eubacterium hallii (Eh); and (2) detecting the relative abundance from step (1) to be lower than a standard control value and determining the individual as having increased risk of ASD.

In a four aspect, a method is provided for assessing risk for autism spectrum disorder (ASD) in a human child. The method comprises these steps: (1) determining, in a stool sample from the child, the level or relative abundance of one or more of the bacterial species set forth in Table 3; (2) determining the level or relative abundance of the same bacterial species in a stool sample from a reference cohort comprising normal and ASD children; (3) generating decision trees by random forest model using data obtained from step (2) and running the level or relative abundance of one or more of the bacterial species from step (1) down the decision trees to generate a risk score; and (4) determining the child with a risk score greater than 0.5 as having an increased risk for ASD and determining the child with a risk score no greater than 0.5 as having no increased risk for ASD.

In some embodiments, the one or more bacterial species comprise or consist of Alistipes indistinctus. In some embodiments, the one or more bacterial species comprise or consist of Alistipes indistinctus, candidate division TM7 single-cell isolate TM7c, and Streptococcus cristatus. In some embodiments, the one or more bacterial species comprise or consist of Alistipes indistinctus, candidate division TM7 single-cell isolate TM7c, Streptococcus cristatus, Eubacterium_limosum, and Streptococcus_oligofermentans.

In a related aspect, the present invention provides a kit for assessing individuals' risk of developing autism spectrum disorder (ASD). The kit comprises reagents for detecting one or more of the bacterial species set forth in Table 1, 2, or 3. In some embodiments, the reagents comprise a set of oligonucleotide primers for amplification of a polynucleotide sequence from any one of the bacterial species set forth in Table 1, 2, or 3. In some embodiments, the amplification is PCR, for example, quantitative PCR.

In a fifth aspect, the present invention provides a method for determining the growth or developmental age of a child. The method comprises these steps: (a) quantitatively determining the relative abundance of one or more bacterial species selected from Table 8 or 9 in a stool sample taken from the child; (b) quantitatively determining the relative abundance of the one or more bacterial species in a stool sample taken from a reference cohort consisting of typically developing children; (c) generating decision trees by random forest model using data obtained from step (b); and (d) running the relative abundances obtained from step (a) down the decision trees from step (b) to generate a developmental age for the child. In some embodiments, the one or more bacterial species comprise Streptococcus gordonii, Enterococcus avium, Eubacterium_sp_3_1_31, Clostridium hathewayi, and Corynebacterium durum. In some embodiments, the one or more bacterial species comprise Streptococcus gordonii, Enterococcus avium, Eubacterium_sp_3_1_31, and Clostridium hatheway. In some embodiments, the one or more bacterial species comprise Streptococcus gordonii, Enterococcus avium, and Eubacterium_sp_3_1_31. In some embodiments, the one or more bacterial species comprise Streptococcus gordonii and Enterococcus avium. In some embodiments, the one or more bacterial species comprise Streptococcus gordonii. In some embodiments, the child is between about 3 to about 6 years old.

In a related aspect, a kit is provided for determining the growth or developmental age of a child. The kit includes a first container containing a first reagent for detecting a first bacterial species set forth in Table 8 or 9 and a second container containing a second reagent for detecting a second and different bacterial species set forth in Table 8 or 9. In some embodiments, the kit includes three or more containers, each of which containing a reagent for detecting a different bacterial species set forth in Table 8 or 9. In some embodiments, the kit includes two or more containers, each of which containing a reagent for detecting a different bacterial species selected from the group consisting of (1) Streptococcus gordonii, Enterococcus avium, Eubacterium_sp_3_1_31, Clostridium hathewayi, and Corynebacterium durum; (2) Streptococcus gordonii, Enterococcus avium, Eubacterium_sp_3_1_31, and Clostridium hatheway; (3) Streptococcus gordonii, Enterococcus avium, and Eubacterium_sp_3_1_31; or (4) Streptococcus gordonii and Enterococcus avium. In some embodiments, the reagents comprise a set of oligonucleotide primers for amplification of a polynucleotide sequence from any one of the bacterial species set forth in Table 8 or 9. In some embodiments, the amplification is PCR, for example, quantitative PCR (qPCR).

In a sixth aspect, the present invention provides a method for promoting growth and development of a child, comprising administering to the child an effective amount of one or more bacterial species selected from Table 8. In some embodiments, the child is between about 3 to about 6 years old in biological age.

In a related aspect, a kit is provided for promoting growth and development of a child. The kit includes a first container containing a first composition comprising (i) an effective amount of one of the bacterial species set forth in Table 8 and a second container containing a second composition comprising (i) an effective amount of another, different one of the bacterial species set forth in Table 8. In some embodiments, the first or second composition comprises processed donor fecal material for FMT. In some embodiments, the first or second composition is formulated for oral administration. In some embodiments, both the first and second compositions are formulated for oral ingestion.

### BRIEF DESCRIPTION OF THE DRAWINGS

FIG. 1. Differential bacterial species between autism spectrum disorder children and typically developing children. The green bars represent species enriched in typically developing children, whereas the red bars represent species enriched in ASD children.

FIG. 2. Receiver operating characteristic (ROC) curve and the area under the curve (AUC) of the machine learning model. AUC of random forest model using: Top 1 marker (red, bottom line)—Alistipes indistinctus. Top 3 markers (green, middle line)-Alistipes indistinctus, candidate division TM7 single-cell isolate TM7c, and Streptococcus cristatus. All 5 markers (dark blue, top line)—Alistipes indistinctus, candidate division TM7 single-cell isolate TM7c, Streptococcus cristatus, Eubacterium_limosum, and Streptococcus_oligofermentans.

FIG. 3. Risk score of a 3-year-old child compared to ASD children and typically developing children using (a) 5 markers: Alistipes indistinctus, candidate division TM7 single-cell isolate TM7c, Streptococcus cristatus, Eubacterium_limosum, and Streptococcus_oligofermentans (b) 3 markers: Alistipes indistinctus, candidate division TM7 single-cell isolate TM7c, Streptococcus cristatus.

FIG. 4. Risk score of a 20-year-old female compared to ASD children and typically developing children using (a) 5 markers: Alistipes indistinctus, candidate division TM7 single-cell isolate TM7c, Streptococcus cristatus, Eubacterium_limosum, and Streptococcus_oligofermentans (b) 3 markers: Alistipes indistinctus, candidate division TM7 single-cell isolate TM7c, Streptococcus cristatus.

FIG. 5. Receiver operating characteristic (ROC) curve and the area under the curve (AUC) of the machine learning model. AUC of random forest model using all 5 markers—Alistipes indistinctus, candidate division TM7 single-cell isolate TM7c, Streptococcus cristatus, Eubacterium_limosum, and Streptococcus_oligofermentans.

FIG. 6. Box plot showing the mean risk score of ASD and TD children in original cohort (64 ASD vs 64 TD children) and independent validation cohort (8 ASD vs 10 TD children).

FIG. 7. Random forest regression was used to identify bacterial species for determining risk of growth and development delay using fecal microbes from 64 typically developing subjects as training cohort. Dotchart of variable importance are shown by % IncMSE (Increased in mean squared error (%)). Red box indicated the top 5 most important bacterial species.

FIG. 8. Age of growth and development of a 3-year-old child was predicted by random forest regression using 5 bacterial markers: Streptococcus gordonii, Enterococcus avium, Eubacterium_sp_3_1_31, Clostridium hathewayi, Corynebacterium durum. Red triangle indicated the predicted age of growth and development of this 3-year-old child. Blue points represented predicted age of growth and development of the 64 typically developing subjects.

FIG. 9. Age of growth and development of a 3-year-old child was predicted by random forest regression using 4 bacterial markers: Streptococcus gordonii, Enterococcus avium, Eubacterium_sp_3_1_31, Clostridium hathewayi. Red triangle indicated the predicted age of growth and development of this 3-year-old child. Blue points represented predicted age of growth and development of the 64 typically developing subjects.

FIG. 10. Age of growth and development of a 3-year-old child was predicted by random forest regression using 3 bacterial markers: Streptococcus gordonii, Enterococcus avium, Eubacterium_sp_3_1_31. Red triangle indicated the predicted age of growth and development of this 3-year-old child. Blue points represented predicted age of growth and development of the 64 typically developing subjects.

FIG. 11. Age of growth and development of a 3-year-old child was predicted by random forest regression using 2 bacterial markers: Streptococcus gordonii, Enterococcus avium. Red triangle indicated the predicted age of growth and development of this 3-year-old child. Blue points represented predicted age of growth and development of the 64 typically developing subjects.

FIG. 12. Age of growth and development of a 3-year-old child was predicted by random forest regression using 1 bacterial markers: Streptococcus gordonii. Red triangle indicated the predicted age of growth and development of this 3-year-old child. Blue points represented predicted age of growth and development of the 64 typically developing subjects.

FIG. 13. Host factors impacted the gut microbiome in children. (A)The effect size of host factors on children gut bacteriome variation. Effect size and statistical significance were determined via PERMANOVA. Only significant host factors were shown, *p<0.05, **p<0.01. (B-C) Heatmaps for correlation between individual host factors and gut bacterial species. Correlation coefficients were calculated through Spearman's (B) and Kendall's (C) correlation coefficient analysis respectively. Statistical significance was determined for all pairwise comparisons. Only statistically significant correlations with absolute coefficient>0.2 were plotted. The color intensity of bottom bar was proportional to the correlation coefficient, where blue indicate positive correlations and yellow indicate inverse correlations.

FIG. 14. Alteration in gut microbiome in Chinese children with ASD. (A) Comparison of fecal bacterial richness between ASD and TD. For box plots, the boxes extend from the 1st to 3rd quartile (25th to 75th percentiles), with the median depicted by a horizontal line. Statistical significance between ASD and TD group was determined by t-test, *p<0.05. (B) Principal coordinate analysis (PCOA) of bacterial community composition in ASD and TD group based on Bray-Curtis dissimilarities, statistical significance was determined by t test, *p<0.05. (C) Comparison of the relative abundance of 5 bacterial species between ASD and TD. The 5 bacterial species markers were identified by Random forest and 10-fold cross-validation. (D) Random forest classifier performance for classifying ASD versus TD microbiome. Receiver operating characteristic (ROC) curves depict trade-offs between RF classifier true and false positive rates as classification stringency varies. AUC values of the training set, test set and validation set represented were given in red, blue and green.

FIG. 15. Gut bacterium-bacterium ecological network in children with ASD versus TD children. Correlations between bacteria-bacteria at the species level in ASD and TD respectively. Correlations between taxa were calculated through Spearman's correlation analysis. Statistical significance was determined for all pairwise comparisons. Only statistically significant correlations with |correlation coefficient|>0.5 were plotted. The correlation network was visualized via Cytoscape (3.8.1). The size of node, corresponding to individual microbial species, is proportional to the number of significant inter-species connections. The color of node indicates the phylum to which the corresponding microbial species belong. The color intensity of connective lines is proportional to the correlation coefficient, where blue lines indicate inverse correlations and red lines indicate positive correlations.

FIG. 16. Functionality alterations in the gut microbiome in ASD. (A) Abundance of pathways related to neurotransmitter biosynthesis in ASD versus TD. The significance was determined by t test and is indicated as *p<0.05. Species contribution to the indicated microbial functionalities, aromatic amino acid (B) and glycine biosynthesis (C), in the gut microbiome of ASD and TD children respectively. In each functional module, the biosynthesis was contributed by a mixture of species (blocks of each stacked bar) in the gut, and each stacked bar represents one of subject metagenomes. (D) Correlations between host factors and abundance of gut microbial functional pathway. Correlations were calculated through Spearman's correlation analysis. Statistical significance was determined for all pairwise comparisons. Only statistically significant correlations with absolute coefficient>0.2 were plotted. The color intensity is proportional to the correlation coefficient, where blue indicates positive correlation and yellow indicates inverse correlation.

FIG. 17. Under-development of age-discriminatory taxa in ASD. (A) Twenty-six species were identified as age-discriminatory bacterial taxa via Random Forest regression of relative abundances of fecal bacterial species against host chronologic age in TD subjects. The age-discriminatory species was ranked in descending order of their importance to the accuracy of the model. Importance was determined based on the percentage increase in mean-squared error of microbiota age prediction when the relative abundance values of each taxon were randomly permuted. The insert shows 5 times tenfold cross-validation error as a function of the number of input bacterial species (blue line). (B) Heatmap of the relative abundances of the 26 age-discriminatory bacterial taxa plotted against the chronologic age spectrum (months) in TD and ASD children respectively. (C) Under-development of the gut microbiome in ASD children versus TD children. Microbiota age prediction model was established as a function of the biological age in TD subjects, and was then employed to predict the microbiome age against their chronological age in ASD.

FIG. 18. Correlations between host factors and the gut bacteriome composition. (A) Redundancy analysis (RDA) of the microbiota composition responding to metadata in ASD and TD. Arrows in the RDA denote the magnitudes and directions of the effect of host factors in shaping the gut microbiome in Chinese children. (B) Comparison of Parabacteroides merdae abundance between children delivered by vagina versus cesarean-section in ASD and TD subjects respectively. Statistical test was performed by Kruskal-Wallis test, * P<0.05, ** P<0.01.

FIG. 19. Differential genera between ASD and TD and prediction model of ASD. (A) Differential bacterial genera at the genus level. LDA scores denote the effect size of the difference in the bacterial abundance between TD and ASD (threshold LDA score>2). Red bars indicate taxa enriched in ASD, and green bars indicate taxa enriched in TD. (B) Risk score of ASD for each participant in discovery and validation sets, respectively. Risk score represent the possibility of randomly generated decision trees that predicted as ASD.

FIG. 20. Gut microbiome functionality alterations in children with ASD. (A) Differential microbial functions in ASD and TD children identified via LefSE. Effect size is shown as LDA score. Only species with LDA score>1 are shown. Red bars indicate functions enriched in ASD, and green bars indicate functions enriched in TD. (B) The abundance of L-serine and glycine biosynthesis pathway, contributed by the species Faecalibacterium prausnitzii, in the gut microbiome of ASD versus TD children. Statistical test was performed by t-test, * P<0.05. (C) The abundance of glutamate synthase-coding genes in the gut microbiome of ASD versus TD children. Statistical test was performed by t-test, * P<0.05.

FIG. 21. Comparison of the relative abundance of three bacterial markers between children with ASD and typically developing children. ASD: autism spectrum disorder; TD: typically developing children.

FIG. 22. Diagnostic performances of bacterial markers in predicting risk of ASD. Receiver operating characteristic (ROC) curve analyses and diagnostic performances of the combined score in distinguishing ASD and typically developing children.

### DEFINITIONS

The term “fecal microbiota transplantation (FMT)” or “stool transplant” refers to a medical procedure during which fecal matter containing live fecal microorganisms (bacteria, fungi, viruses, and the like) obtained from a healthy individual is transferred into the gastrointestinal tract of a recipient to restore healthy gut microflora that has been disrupted or destroyed by any one of a variety of medical conditions, for example, autism spectrum disorder (ASD). Typically, the fecal matter from a healthy donor is first processed into an appropriate form for the transplantation, which can be made through direct deposit into the lower gastrointestinal tract such as by colonoscopy, or by nasal intubation, or through oral ingestion of an encapsulated material containing processed (e.g., dried and frozen or lyophilized) fecal material.

The term “inhibiting” or “inhibition,” as used herein, refers to any detectable negative effect on a target biological process, such as RNA/protein expression of a target gene, the biological activity of a target protein, cellular signal transduction, cell proliferation, and the like. Typically, an inhibition is reflected in a decrease of at least 10%, 20%, 30%, 40%, 50%, 60%, 70%, 80%, 90% or greater in the target process (e.g., growth or proliferation of a microorganism of certain species, for example, one or more of the bacterial species shown in Table 2), or any one of the downstream parameters mentioned above, when compared to a control. “Inhibition” further includes a 100% reduction, i.e., a complete elimination, prevention, or abolition of a target biological process or signal. The other relative terms such as “suppressing,” “suppression,” “reducing,” “reduction,” “decrease,” “decreasing,” “lower,” and “less” are used in a similar fashion in this disclosure to refer to decreases to different levels (e.g., at least 10%, 20%, 30%, 40%, 50%, 60%, 70%, 80%, 90% or greater decrease compared to a control level, i.e., the level before suppression) up to complete elimination of a target biological process or signal. On the other hand, terms such as “activate,” “activating,” “activation,” “increase,” “increasing,” “promote,” “promoting,” “enhance,” “enhancing,” “enhancement,” “higher,” and “more” are used in this disclosure to encompass positive changes at different levels (e.g., at least 10%, 20%, 30%, 40%, 50%, 60%, 70%, 80%, 90%, 100%, 200%, or greater such as 3, 5, 8, 10, 20-fold increase compared to a control level (before activation), for example, the control level of one or more of the bacterial species shown in Table 1) in a target process or signal. In contrast, the term “substantially the same” or “substantially lack of change” indicates little to no change in quantity from a comparison basis (such as a standard control value), typically within ±10% of the comparison basis, or within ±5%, 4%, 3%, 2%, 1%, or even less variation from the comparison basis.

“Standard control” as used herein refers to a value corresponding to either the average level of a pre-selected bacterial species found in a particular type of samples (e.g., stool samples) obtained from individuals who did not suffer from ASD or developmental delay or a composite score calculated from the average levels of multiple bacterial species found in the type of samples taken from such individuals. For example, for the purpose of examining the risk of ASD in a child, a “standard control” value is established to provide a cut-off value to indicate whether or not the child being examined has an elevated risk for ASD. In order for a “standard control” to be properly established, a sufficient number of individuals (e.g., at least 10, 12, 15, 20, 24 or more individuals) must be included in the control group to provide samples for determination of the average level(s) of one or more pre-selected bacterial species or the composite score calculated from the levels of multiple bacterial species representative of the risk for ASD.

The term “anti-bacterial agent” refers to any substance that is capable of inhibiting, suppressing, or preventing the growth or proliferation of bacterial species, respectively, especially those of shown in Table 2. Known agents with anti-bacterial activity include various antibiotics that generally suppress the proliferation of a broad spectrum of bacterial species as well as agents such as antisense oligonucleotides, small inhibitory RNAs, and the like that can inhibit the proliferation of specific bacterial species. The term “anti-bacterial agent” is similarly defined to encompass both agents with broad spectrum activity of killing virtually all species of bacteria and agents that specifically suppress proliferation of target bacteria species. Such specific anti-bacterial agent may be short polynucleotide in nature (e.g., a small inhibitory RNA, microRNA, miniRNA, lncRNA, or an antisense oligonucleotide) that is capable of disrupting the expression of a key gene in the life cycle of a target bacterial species and is therefore capable of specifically suppressing or eliminating the species only without substantially affecting other closely related bacterial species.

“Percentage relative abundance,” when used in the context of describing the presence of a particular bacterial species (e.g., any one of those shown in any one of Tables 1-11) in relation to all bacterial species present in the same environment, refers to the relative amount of the bacterial species out of the amount of all bacterial species as expressed in a percentage form. For instance, the percentage relative abundance of one particular bacterial species can be determined by comparing the quantity of DNA specific for this species (e.g., determined by quantitative polymerase chain reaction) in one given sample with the quantity of all bacterial DNA (e.g., determined by quantitative polymerase chain reaction (PCR) and sequencing based on the 16s rRNA sequence) in the same sample.

“Absolute abundance,” when used in the context of describing the presence of a particular bacterial species (e.g., any one of those shown in Tables 1-11) in the feces, refers to the amount of DNA derived from the bacterial species out of the amount of all DNA in a fecal sample. For instance, the absolute abundance of one bacterium can be determined by comparing the quantity of DNA specific for this bacterial species (e.g., determined by quantitative PCR) in one given sample with the quantity of all fecal DNA in the same sample.

“Total bacterial load” of a fecal sample, as used herein, refers to the amount of all bacterial DNA, respectively, out of the amount of all DNA in the fecal sample. For instance, the absolute abundance of bacteria can be determined by comparing the quantity of bacteria-specific DNA (e.g., 16s rRNA determined by quantitative PCR) in one given sample with the quantity of all fecal DNA in the same sample.

As used herein, the term “autism spectrum disorder (ASD),” refers to a condition related to brain development that impacts how a person perceives and socializes with others, resulting in difficulties in social interaction and communication. ASD begins in early childhood and eventually causes problems in the suffers' inability to function properly in society-socially, in school, and at work. The term “spectrum” in autism spectrum disorder refers to the wide range of symptoms and severity. ASD includes conditions that were previously considered separate, such as autism, Asperger's syndrome, childhood disintegrative disorder, and an unspecified form of pervasive developmental disorder. The disorder also includes limited and repetitive patterns of behavior.

The term “treat” or “treating,” as used in this application, describes an act that leads to the elimination, reduction, alleviation, reversal, prevention and/or delay of onset or recurrence of any symptom of a predetermined medical condition. In other words, “treating” a condition encompasses both therapeutic and prophylactic intervention against the condition, including facilitation of patient recovery from the condition.

The term “effective amount,” as used herein, refers to an amount of a substance that produces a desired effect (e.g., an inhibitory or suppressive effect on the growth or proliferation of one or more detrimental bacterial species (e.g., the bacterial species shown in Table 2) for which the substance (e.g., an anti-bacterial agent) is used or administered. The effects include the prevention, inhibition, or delaying of any pertinent biological process during bacterial proliferation to any detectable extent. The exact amount will depend on the nature of the substance (the active agent), the manner of use/administration, and the purpose of the application, and will be ascertainable by one skilled in the art using known techniques as well as those described herein. In another context, when an “effective amount” of one or more beneficial or desirable bacterial species (e.g., those listed in Table 1) are artificially introduced into a composition intended to be introduced into the gastrointestinal tract of a patient, e.g., to be used in FMT, it is meant that the amount of the pertinent bacteria being introduced is sufficient to confer to the recipient health benefits such as reduced recovery time or reduced needs for therapeutic intervention for a pertinent disorder such as ASD, including but not limited to medication (such as antipsychotic drugs and antidepressants) and any of the variety of therapies such as behavior and communication therapy, educational therapy, family therapy, speech or physical therapy, and the like.

The term “growth/development age,” as used herein, refers to a child's developmental stage that is expressed in time units and assessed based on the status/profile of microorganism present in his or her GI tract. A comparison between a child's biological (birth) age and growth/development age reflects whether or not the child's growth and development is consistent with his or her birth or chronological age or “age-appropriate.”

As used herein, the term “about” denotes a range of value that is +/−10% of a specified value. For instance, “about 10” denotes the value range of 9 to 11 (10+/−1).

### DETAILED DESCRIPTION OF THE INVENTION

I. Introduction

The invention provides a novel approach for assessing the risk for developing autism spectrum disorder (ASD) among children, for assessing the growth or development age among children, as well as for treating ASD symptoms. During their studies, the present inventors discovered that the presence and relative abundance of certain bacterial species alter significantly in the gastrointestinal tract of patients due to ASD, with increase or decrease of particular species correlating with disease severity. For example, the presence of bacterial species shown in Table 2 is found to be at an elevated level in the GI tract of ASD children, whereas the presence of bacterial species such as those shown in Table 1 have been found to be at a reduced level in the GI tract of ASD children. On the other hand, the level or relative abundance of certain bacterial species (such as those shown in Table 3) in children's stool samples has been observed to correlate with likelihood of children developing ASD at a later time. Lastly, the inventors discovered that the microorganism presence and profile within a child's GI tract evolves as the child progresses along with his growth and development process. Thus, the results of these latest discoveries provide useful tools for treating ASD symptoms, for assessing ASD risk among children, for guiding the necessary treatment such as medication and/or therapies described herein for children who have been identified as at high risk of ASD or are exhibiting symptoms of ASD, as well as for assessing a child's growth and developmental age to determine whether he is appropriate in his development process in relation to his biological or birth age, which can then facilitate subsequently determining whether or not certain treatment is needed, for example, supplementary administration of certain bacterial species found to be deficient in the GI tract of the child for the purpose of promoting his growth and development.

II. FMT Donor/Recipient Selection and Preparation

ASD children suffer from a disrupted state of GI tract microflora are considered as recipients for FMT treatment in order to restore the normal healthy profile for microorganisms. As revealed by the present inventors, the presence or risk of ASD tends to lead to a depressed level of bacterial species such as those shown in Table 1, a FMT donor whose fecal material contains an higher than average level of one or more of these bacterial species is favored as particularly advantageous for this purpose. For example, a desirable donor may preferably have higher than about 0.01%, 0.02%, 0.05%, 0.10%, 0.20%, 0.40%, 0.50%, 0.60%. 0.80%, 1.0%, 2.0%, 3.0%, 4.0%, 5.0%, 6.0%, 7.0%, 8.0%, 8.5%, 9.0%, or higher of total bacteria in relative abundance for each of these bacterial species in his stool sample.

On the other hand, ASD children have abnormally high level of the bacterial species listed in Table 2. Thus, to restore their normal and healthy GI bacterial profile, FMT is appropriate using fecal material donated from a healthy person whose level of these bacterial species (in Table 2) in the stool sample is either naturally low or artificially depressed, for example, by the use of a specific anti-bacterial agent that specifically kills or suppresses certain target bacterial species without significantly impacting other bacterial species. Preferably, each of these bacterial species should have no more than about 0.01%, 0.02%, 0.05%, 0.07%, 0.08%, 0.10%, 0.13%, 0.15%, 0.20%, 0.25%, 0.30%, 0.50%, 0.70% or higher of total bacteria in relative abundance in the fecal material before being processed for use in FMT.

Fecal matter used in FMT is obtained from a healthy donor and then processed into appropriate forms for the intended means of delivery in the upcoming FMT procedure. While a healthy individual from the same family or household often serves as donor, in practicing the present invention the donor microorganism profile is an important consideration and may favor the choice of an unrelated donor instead. The process of preparing donor material for transplant includes steps of drying, freezing or lyophilizing, and formulating or packaging, depending on the precise route of delivery to recipient, e.g., by oral ingestion or by rectal deposit.

In preparation for FMT treatment, an intended recipient, e.g., a patient who has been diagnosed with ASD or who was deemed to have an increased risk of developing ASD but has not yet exhibited any definitive symptoms for the disease (e.g., has family history or other known risk factors for ASD), may first receive a treatment to suppress bacterial level in his GI tract prior to FMT. The treatment may involve administration of an anti-bacterial agent, either a broad spectrum antibiotic or a specific anti-bacterial agent, to eliminate or reduce the level of undesirable bacterial species that has risen due to the ASD presence or risk, such as one or more of the bacteria named in Table 2.

Various methods have been reported in the literature for determining the levels of all bacterial species in a sample, for example, amplification (e.g., by PCR) and sequencing of bacterial polynucleotide sequence taking advantage of the sequence similarity in the commonly shared 16S rRNA bacterial sequences. On the other hand, the level of any given bacterial species may be determined by amplification and sequencing of its unique genomic sequence. A percentage abundance is often used as a parameter to indicate the relative level of a bacterial species in a given environment.

III. Treatment Methods by Modulating Bacterial Level

The discovery by the present inventors reveals the direct correlation between ASD and the increase or decrease of certain bacterial species (e.g., those shown in Table 1 or 2) in ASD children's gut. This revelation enables different methods for treating ASD symptoms, especially for aiding ASD children to benefit from different treatment regimens such as medication and/or various therapies, by adjusting or modulating the level of these bacterial species in these patient's GI tract via, e.g., an FMT procedure, to either deliver to the patients' GI tract an effective amount of one or more of the bacterial species of those shown in Table 1 or to decrease the level of one or more bacterial species listed in Table 2, e.g., by delivering an anti-bacterial agent to suppress the target bacterial species.

When a proposed FMT donor whose stool is tested and found to contain an insufficient level of one or more of the beneficial bacterial species such as those shown in Table 1 (e.g., each is less than about 0.01%, 0.05%, 0.10%, 0.20%, 0.40%, 0.50%, 0.80%, 1.0%, 2.0%,3.0%,4.0%,5.0%,6.0%,7.0%, or 8.0% of total bacteria in the stool sample), the proposed donor is deemed as an unsuitable donor for FMT intended to treat ASD symptoms or to reduce a recipient's (e.g., a child's) risk for developing ASD in the future, he may be disqualified as a donor in favor of anther individual whose stool sample exhibits a more favorable bacterial profile, and his fecal material should not be immediately used for FMT due to the lack of prospect of conferring such beneficial health effects unless the stool material is adequately modified. In these cases of expected lack of health benefits from FMT treatment can be readily improved in view of the inventors' discovery, for example, one or more of the bacterial species such as those shown in Table 1 may be introduced from an exogenous source into a donor fecal material so that the level of the bacterial species in the fecal material is increased (e.g., to reach at least about 0.01%, 0.02%, 0.05%, 0.10%, 0.20%, 0.40%, 0.50%, 0.60%. 0.80%, 1.0%, 2.0%, 3.0%, 4.0%, 5.0%, 6.0%, 7.0%, 8.0%, 8.5%, 9.0%, or 10% of total bacteria in the fecal material) before it is processed for use in FMT for the treatment of ASD symptoms or for reducing ASD risk in a child. Pre-treatment schemes with similarly intended goals can be employed to prepare patients who are soon to receive FMT treatment in order to maximize their potential to receive health benefits such as those stated above and herein.

As an alternative, the beneficial bacterial species (one or more of those shown in Table 1) may be obtained from a bacterial culture in a sufficient quantity and then formulated into a suitable composition, which is without any fecal material taken from a donor, for delivery into an ASD patient's gut. Similar to FMT, such composition can be introduced into a patient by oral, nasal, or rectal administration.

On the other hand, certain bacterial species (e.g., those in Table 2) are found to rise in their relative abundance as a result of the presence of ASD or risk of ASD. Thus, ASD patients or those at heightened risk for ASD are treated to reduce the level of these bacterial species in order to ameliorate the patients' symptoms related to the illness. There are several options to reduce the level of these bacterial species: first, the patient may be given a specific anti-bacterial agent to specifically kill or suppress the targeted bacterial species, thereby lowering the abnormally high level of these bacteria.

Second, the patient may be first given an anti-bacterial agent, such as a broad spectrum antibiotic to kill or suppress all bacterial species, or a specific anti-bacterial agent to specifically kill or suppress the targeted bacterial species; then a composition may be administered to the patient (e.g., by FMT) to introduce a well-balanced mixed bacterial culture into the GI tract of the patient.

Each of these options can be performed in one combined step to achieve the first and second treatment method goals, i.e., to increase the level of certain bacterial species (such as one or more of those shown in Table 1) and to decrease the level of certain other bacterial species (for example, one or more of those listed in Table 2), using one single composition (such as processed fecal material from an FMT donor) containing the pertinent bacterial species within the appropriate ratio range to one another.

Immediately upon completion of the step of introducing an effective amount of the desired bacterial species into a patient's GI tract (e.g., via an FMT procedure) and/or the step of suppressing undesirable bacterial level, the recipient may be further monitored by continuous testing of the level or relative abundance of the bacterial species in the stool samples on a daily basis for up to 5 days post-procedure while the clinical symptoms of ASD being treated as well as the general health status of the patient are also being monitored in order to assess treatment outcome and the corresponding levels of relevant bacteria in the recipient's GI tract: the level of bacterial species (one or more of those shown in Table 1) may be monitored in connection with observation of health benefits achieved such as improvement in behavior, language or social skills.

IV. Assessing Disease Severity and Corresponding Treatment

The present inventors also discovered that the altered level of certain bacterial species can indicate the presence or risk of ASD: they revealed the correlation between reduced level of certain bacterial species (e.g., those shown in Table 1) in human children's stool samples and the likelihood of a later diagnosis of ASD in these children. Similarly, a correlation between increased level of certain other bacterial species (e.g., those shown in Table 2) in a child's GI tract and the likelihood of the child later developing ASD has been established. Further, the level or relative abundance of certain bacterial species (such as one or more of the species shown in Table 3) have been revealed to indicate a subject's risk for later developing ASD when properly calculated using certain specified mathematic tools.

For example, when stool samples taken from two or more children, the level or relative abundance of bacterial species in Table 1 or 2 in the samples may be determined, for example, by PCR especially quantitative PCR. For the bacterial species listed in Table 1, a lower level found in a child's stool sample indicates a higher likelihood for the presence or increased risk of ASD in the child; conversely, for the bacterial species listed in Table 2, a higher level found in a child's stool sample indicates a higher likelihood of the presence or risk for ASD in the child. In the event that the level of multiple species are measured and compared, the rick determination is made based on the indication from the majority of the pertinent bacterial species measured.

Once the ASD risk assessment is made, for example, a child is deemed to have ASD or is at an increased risk of later developing ASD, appropriate treatment steps can be taken as a measure to address the heightened risk for the child. For example, the child may be given medication such as antipsychotic and/or antidepressant drugs or may be given therapies such as those specifically designed to address behavioral problems and/or to improve language, communication, or social skills

V. Assessing Growth/Development Age in Children

The present inventors have in addition revealed that the profile of bacterial species present in a child's gastrointestinal tract continues to evolve as the child continues to develop as a part of the normal growth process. Thus, the results disclosed therein further allows one to devise an effective and accurate means for assessing children's developmental age based on the levels of certain relevant gut bacterial species using the methods described herein. More specifically, a stool sample is first taken from the child who is being tested for his growth or development age. The level or relative abundance of a plurality of pre-selected bacterial species (such as the bacterial species shown in Table 8 or 9) are then quantitatively determined using methods known in the pertinent field or described herein. Using the levels of these bacterial species one can subsequently calculate the child's development age using mathematic tools specifically described in this disclosure.

Once a child's growth or developmental age is determined using the method of this invention, if needed the child may be given appropriate treatment for the purpose of promoting his growth or development. For example, if a child's development age is found to be well behind his biological age, e.g., more than about 6 or 9 or 12 months behind his biological age, or more than about 10%, 20%, 25%, 33%, or even 50% behind his biological age, he may be given treatment by way of administration of an effective amount of one or more of the bacterial species named in Table 8 or 9 and found to be deficient in his gastrointestinal tract. One method of treatment is FMT, e.g., oral administration or direct deposit of pre-processed material enriched with the desired bacterial species.

VI. Kits and Compositions for Use in ASD Treatment

The present invention provides novel kits and compositions that can be used for alleviating the symptoms and conferring health benefits in the therapeutic and/or prophylactic treatment of ASD, including facilitation of patient improvement by way of conventional therapies designed for treating ASD. For example, a kit is provided that comprises a first container containing a first composition comprising (i) an effective amount of one or more of the bacterial species set forth in Table 1 or 14, or (ii) an effective amount of an anti-bacterial agent that suppresses growth of one or more of the bacterial species set forth in Table 2 or 13, and a second container containing a second composition comprising an effective amount of a medicine known for use in the treatment of ASD (such as an antipsychotic or antidepressant drug). In some variations, the kit may contain two or more compositions each of which comprises an effective amount of (1) one or more of the beneficial bacterial species of Table 1 or 14, (2) an anti-bacterial agent, and (3) a medicine for treating ASD, either alone or in any combination.

In some cases, the first composition comprises a fecal material from a donor, which has been processed, formulated, and packaged to be in an appropriate form in accordance with the delivery means in the FMT procedure, which may be by direct deposit in the recipient's lower gastrointestinal track (e.g., wet or semi-wet form) or by oral ingestion (e.g., frozen, dried/lyophilized, encapsulated). Alternatively, the first composition may not contain any donor fecal material but is an artificially mix containing the preferred bacterial species, such as one or more of the bacterial species set forth in Table 1 or 14, at an appropriate ratio and quantity. Further, the first composition may contain an adequate amount of an anti-bacterial agent that suppresses growth of one or more of the bacterial species set forth in Table 2 or 13. The anti-bacterial agent may be a broad-spectrum anti-bacterial agent in some cases; or in other cases it may be a specific anti-bacterial agent targeting the specific bacterial species only (e.g., those in Table 2 or 13): it may be a short polynucleotide, e.g., a small inhibitory RNA, microRNA, miniRNA, lncRNA, or an antisense oligonucleotide, that is capable of specifically targeting one or more of predetermined bacterial species without significantly affecting other closely related bacterial species.

In other cases, the first composition may be a composition (e.g., a processed FMT donor fecal material) comprising the preferred bacterial species (such as one or more of the bacterial species set forth in Table 1 or 14) at an appropriate ratio and quantity along with a specific anti-bacterial agent targeting the specific bacterial species only (e.g., those named in Table 2 or 13). The first composition is formulated and packaged in accordance with its intended means of delivery to the patient, for example, by oral ingestion, nasal delivery, or rectal deposit.

The second composition in some cases may comprises an adequate or effective amount of a therapeutic agent effective for treating ASD, for example, an antipsychotic or antidepressant drug. The composition is formulated for the intended delivery method of the prebiotic or therapeutic agent(s), for example, by injection (intravenous, intraperitoneal, intramuscular, or subcutaneous injection) or by oral/nasal administration or by local deposit (e.g., suppositories).

The first and second compositions are often kept separately in two different containers in the kit. In some cases, the composition for increasing the level of certain bacterial species (such as one or more of the bacterial species set forth in Table 1 or 14) and the composition for suppressing other bacterial species (e.g., one or more of those listed in Table 2 or 13) may be combined to form a single composition for administration to the patient together, for example, by oral or local delivery, at the same time. In some cases, the first and second compositions may be combined in a single composition so that they can be administered to the patient together, for example, by oral or local delivery, at the same time.

Moreover, a kit is provided for the quantitative detection of one or more bacterial species such as the bacterial species set forth in Tables 1, 2, 13, and 14. The kit comprises reagents for quantitative detection of each of the bacterial species, for example, such reagents may comprise a set of oligonucleotide primers for the amplification, such as PCR especially quantitative PCR, of a polynucleotide sequence derived from, and preferably unique to, each one of the pertinent bacterial species (such as any one or more of the bacterial species set forth in Tables 1-3), especially those set forth in Tables 1, 2, 13, and 14.

In addition, the present invention provides kits and compositions for assessing a child's growth or development age as well as for promoting or enhancing a child's growth or development. Typically, a kit for determining developmental age of a child includes a first container containing a first reagent for detecting a first bacterial species set forth in Table 8 or 9 and a second container containing a second reagent for detecting a second (different from the first) bacterial species set forth in Table 8 or 9. For example, the kit may include three or more containers each of which containing a reagent for detecting a different bacterial species set forth in Table 8 or 9. As another example, the kit may include two or more containers each of which containing a reagent for detecting a different bacterial species selected from any of the following groups consisting of (1) Streptococcus gordonii, Enterococcus avium, Eubacterium_sp_3_1_31, Clostridium hathewayi, and Corynebacterium durum; (2) Streptococcus gordonii, Enterococcus avium, Eubacterium_sp_3_131, and Clostridium hatheway; (3) Streptococcus gordonii, Enterococcus avium, and Eubacterium_sp_31_31; or (4) Streptococcus gordonii and Enterococcus avium. The reagents included in the kit for detection of a pre-selected bacterial species may include a set of oligonucleotide primers for amplification of a polynucleotide sequence from (and preferably unique to) the bacterial species, e.g., any one of the bacterial species set forth in Table 8 or 9. A frequently used method of amplification is PCR, such as quantitative PCR (qPCR).

A kit for promoting growth and development of a child (e.g., a child of about 3 to about 6 years of biological or birth age) by way of administering to the child an effective amount of one or more bacterial species selected from Table 8 typically includes a first container containing a first composition comprising (i) an effective amount of one of the bacterial species set forth in Table 8 and a second container containing a second composition comprising (i) an effective amount of another (and different from the first) one of the bacterial species set forth in Table 8. In exemplary embodiments, the first and/or second composition(s) may be or include a processed donor fecal material for FMT. Either or both of the first and second compositions may be formulated for oral administration, for example, to be used in an FMT process. All compositions described herein may contain one or more physiologically acceptable excipients or carriers in addition to the active components.

### EXAMPLES

The following examples are provided by way of illustration only and not by way of limitation. Those of skill in the art will readily recognize a variety of non-critical parameters that could be changed or modified to yield essentially the same or similar results.

### Example I: Gut Bacterial Profile in Ads and Normal Children

### Background

The present inventors studied changes in gut microbiota due to the presence or risk for autism spectrum disorder (ASD) by comparing the profile of bacterial species present in the gastrointestinal tract of autistic children with that of developmentally normal children. The bacterial species that have been found to be present at a decreased level or relative abundance in autistic children, e.g., any one of those set forth in Table 1, and the bacterial species that have been found to be present at an increased level or relative abundance in autistic children, e.g., any one of those set forth in Table 2, can be quantitatively measured to assess an individual's risk of later developing ASD. On the other hand, these bacterial species may be subject to modulation of their level or relative abundance in order to treat ASD by alleviating at least some of its symptoms.

### Methods

### Cohort Description and Study Subjects

A total of 128 Chinese children (aged between 3 and 6 years) were recruited, including 64 children with autism spectrum disorder (ASD) and 64 typically developing children. There were more male (83%) than female. Majority of case were diagnosis with ASD at around 3 years old. The study was approved by The Joint Chinese University of Hong Kong, New Territories East Cluster Clinical Research Ethics Committee (The Joint CUHK-NTEC CREC, CREC Ref. No: 2016.607). All subjects consented to donate fecal samples and to the questionnaire investigation, where written informed consents were obtained. Fecal samples from the study subjects were stored at −80° C. for downstream microbiome analyses. Children diagnosed with ASD by pediatrician or clinical psychologist according to the standard of the fourth or fifth edition of the Diagnostic and Statistical Manual of Mental Disorders (DSM-IV or DSM-V) were included. Children without ASD, delays in motor and language development, as well as behaviors as reported by their parents, and those do not have first-degree relatives with ASD were included as typically developing children.

### Fecal DNA Extraction and DNA Sequencing

Fecal bacterial DNA was extracted by Maxwell® RSC PureFood GMO and Authentication Kit (Promega) with modifications to increase the yield of DNA. Approximately 100 mg from each stool sample was pretreated: stool sample suspended in 1 ml ddH₂O and pelleted by centrifugation at 13,000×g for 1 min. Washed sample was added 800ul TE buffer (PH 7.5), 16ul beta-Mercaptoethanol and 250U lyticase sufficiently mixed and digestion at 37° C. for 90 minutes. Pelleted by centrifugation at 13,000×g for 3 minutes.

After pretreatment, precipitate was re-suspended in 800ul CTAB buffer (Maxwell® RSC PureFood GMO and Authentication Kit following manufacturer's instructions) and mixed well. After samples were heated at 95° C. for 5 minutes and cooled down, nucleic acid was released from the samples by vortexing with 0.5 mm and 0.1 mm beads at 2850 rpm for 15 minutes. Following this, 40ul Proteinase K and 20ul RNase A were added and nucleic acid digested at 70° C. for 10 minutes. Finally, supernatant was obtained after centrifugation at 13,000×g, 5 minutes and placed in a Maxwell® RSC instrument for DNA extraction. The extracted fecal DNA was used for ultra-deep metagenomics sequencing via Ilumina Novaseq 6000 (Novogene, Beijing, China).

### Quality Control of Raw Sequences

Raw sequence reads were trimmed by Trimmomatic¹(v0.38) firstly and then separation of non-human reads from contaminant host reads. There were some steps to acquire clean reads: 1) Remove adapters; 2) Scan the read with a 4-base wide sliding window, removing reads when the average quality per base drop below 20; 3) Drop reads below the 50 bases long. T rimmed sequence reads were mapped to human genome (Reference database: GRCh38 p12) by KneadData (v0.7.2) to remove reads originated from the host. Pair-end two reads were concatenated together.

### Analysis of the Bacterial Microbiome

Profiling of the composition of bacterial communities was performed on metagenomic trimmed reads via MetaPhlAn2 (v2.7.5)². Mapping reads to clade-specific markers gene and annotation of species pangenomes was done through Bowtie2 (v2.3.4.3)³. The output table contained bacterial species and its relative abundance in different levels, from kingdom to strain level. The resulting data were analyzed in R v3.6.1 using tidyverse (v1.2.1)⁴, ggpubr (v0.2, website: github.com/kassambara/ggpubr) and phyloseq (v1.24.2)⁵. Human gut bacteria composition and defined the differential bacterial species were compared between children with autism spectrum disorder (ASD) and typically developing children via Linear discriminant analysis effect size (LEfSe) analysis⁶.

### Machine Learning Model

Random forest (RF) was chosen to build ASD versus typically developing children prediction model using fecal microbes because of its superior performance for classification with binary features. Random Forest⁷ is one of the most popular approaches in metagenomic data analysis to identify the discriminative features and build prediction models. As a widely used ensemble learning algorithm, Random Forest consists of a series of classification and regression trees (CARTs) to form a strong classifier. A subset of data randomly sampled from the original dataset with replacement is known as bootstrap sampling, applying to build the trees. When the training dataset for the current tree is drawn by the bootstrap method,

$$1 - {( 1 - \frac{1}{N} )}^{N}$$

observations are left out from the overall dataset. With infinite N, there are 36.8% data not occurred in the training samples called out-of-bag (OOB) observations, which would not be used for constructing the trees. In addition, extra randomness introduced to the random forest as each decision tree splits nodes based on a random subset of features selected from the overall features. The features with the least Gini (Gini are used to evaluate the purity of the node) would be utilized to split the nodes in each iteration to generate the trees. With different subsets of data and features, the algorithm is able to train different trees and obtain the final classification by averaging the result from the tree models. In addition to the prediction model, Random Forest has the capability to assess the importance of variables⁸. The OOB observations are used to estimate the classification error for each tree in the forest. To measure the importance of a given variable, the values of the variable in the OOB data are randomly altered, and then the changed OOB data is used to generate new predictions. The difference of the error rate between the altered and the original OOB observations divided by the standard error is calculated as the importance of a variable. To classify a new sample, the features of the sample passed down to each tree to estimate the probability for classification. The Random Forest used the average probability of all trees to determine the final result of the classification.

A total of 64 children with ASD patients and 64 typically developing children were included as the discovery cohort for modeling. The importance value of each species to the classification model was evaluated by recursive feature elimination. According to descending importance value, the selected species were added one by one to the random forest model if its Pearson correlation value with any already existing probe in the model was <0.7. Each time a new feature was added to the model, the performance of the model was re-evaluated using 10-fold cross-validation. These models were compared in terms of binary classifiers with Area Under the Curve (AUC) in Receiver Operating Characteristic (ROC) curves. The final model was chosen when best accuracy and kappa were achieved. These analysis was done using R packages randomForest v4.6-14⁷ and pROC v1.15.3⁹.

### Results

### Gut Bacterial Profile is Different Between Autism Spectrum Disorder Children and Typically Developing Children

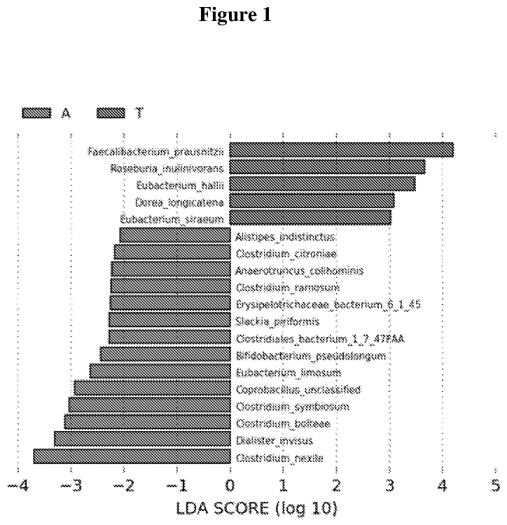

With LEfSe analysis, the species Faecalibacterium prausnitzii, Roseburia inulinivorans, Eubacterium hallii, Dorea longicatena, Eubacterium siraeum (FIG. 1, Table 1) were found to be present in higher relative abundance in typically developing children than in children with ASD. In contrast, the species Clostridium nexile, Dialister invisus, Clostridium bolteae, Clostridium symbiosum, Eubacterium limosum, Clostridiales bacterium_1_7_47FAA, Slackia piriformis, Erysipelotrichaceae_bacterium_6_1_45, Clostridium ramosum, Anaerotruncus colihominis, Clostridium citroniae, Alistipes indistinctus (FIG. 1, Table 2) were enriched in children with ASD when compared to typically developing children.

TABLE 1
Bacterial Species Enriched in Typically Developing Children
Compared to Children with Autism Spectrum Disorder

| | | Cut-off |
|---|---|---|
| | | (relative |
| Bacterial Species | NCBI: txid | abundance) |
| Faecalibacterium prausnitzi | 853 | 8.38% |
| Roseburia inulinivorans | 360807 | 0.58% |
| Eubacterium hallii | 1263078 | 0.40% |
| Dorea longicatena | 88431 | 0.01% |
| Eubacterium siraeum | 39492 | 0.01% |

TABLE 2
Bacterial Species Enriched in Children with Autism Spectrum
Disorder Compared to Typically Developing Children

| | | Cut-off |
|---|---|---|
| | | (relative |
| Bacterial Species | NCBI: txid | abundance) |
| Clostridium nexile | 1263069 | 0.13% |
| Dialister invisus | 218538 | 0.01% |
| Clostridium bolteae | 997896 | 0.10% |
| Clostridium symbiosum | 411472 | 0.07% |
| Eubacterium limosum | 1736 | 0.01% |
| Clostridiales bacterium_1_7_47FAA | 457421 | 0.02% |
| Clostridium ramosum | 1547 | 0.01% |
| Anaerotruncus colihominis | 445972 | 0.01% |
| Clostridium citroniae | 358743 | 0.01% |
| Alistipes indistinctus | 626932 | 0.01% |

Bacteria listed in Table 1 and Table 2 can be used in different combinations to determine the risk of ASD. For example, the relative abundance can be determined using as a panel of qPCR primer or by metagenomics sequencing to calculate the risk.

Furthermore, bacteria listed in Table 1 can be administered to children with ASD or at risk for developing ASD to ameliorate symptoms of ASD or reduce risk for later developing ASD. Conversely, bacteria listed in Table 2 can be targeted for suppression in children with ASD or at risk for developing ASD to ameliorate symptoms of ASD or reduce risk for later developing ASD.

### Machine Learning Model for Prediction of ASD

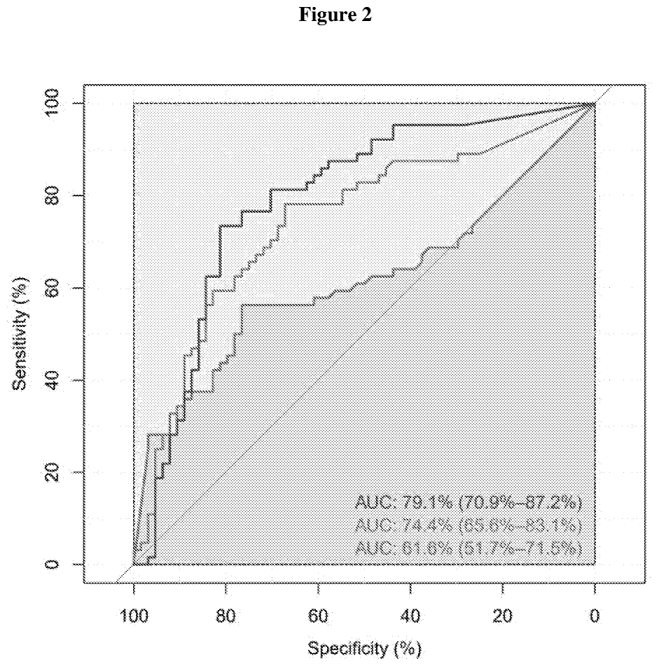

Five bacterial markers were used in the machine learning model, including Alistipes indistinctus, candidate division_TM7 single_cell_isolate_TM7c, Streptococcus cristatus, Eubacterium_limosum, Streptococcus_oligofermentans (Table 3). The final models using these 5 markers has an Area Under the Curve (AUC) in Receiver Operating Characteristic (ROC) curves of 79.1% (FIG. 2).

TABLE 3
Bacterial Species Included in the Machine
Learning Model for Prediction of ASD

| Bacterial Species | NCBI: txid |
|---|---|
| Alistipes indistinctus | 626932 |
| candidate division TM7 single-cell isolate TM7c | 447456 |
| Streptococcus cristatus | 45634 |
| Eubacterium_{—}limosum | 1736 |
| Streptococcus_{—}oligofermentans | 45634 |

To determine the risk of ASD in a subject, the following steps will be carried out:
(1) Obtain a set of training data by determine the relative abundance of species selected from Table 3 in a cohort of typically developing children and patients with ASD.
(2) Determine the relative abundance of these species in the subject who is being tested for his risk of ASD.
(3) Compare the relative abundance of these species in the subject with the training data using random forest model.
(4) Decision trees will be generated by random forest from the training data. The relative abundances will be run down the decision trees and generate a risk score. If at least 50% trees in the model consider the child has autism, the child being tested is deemed to have an increased risk for ASD. If less than 50% trees in the model consider the child as typically developing children, the child being tested is deemed to not have an increased risk for ASD.

In performing the above step (1), the bacterial species selected from Table 3 should comprise of (a) Alistipes indistinctus (top 1 species; AUC: 61.6%; FIG. 2); (b) Alistipes indistinctus, candidate division TM7 single-cell isolate TM7c, and Streptococcus cristatus (top 3 species; AUC: 74.4%; FIG. 2); or (c) Alistipes indistinctus, candidate division TM7 single-cell isolate TM7c, Streptococcus cristatus, Eubacterium_limosum, and Streptococcus_oligofermentans (all 5 species; AUC 79.1%; FIG. 2).

### Study 1

The relative abundance of 5 species listed in Table 3 from 64 children with ASD and 64 typically developing children was determined by metagenomics sequencing and taxonomy assigned as described in methods (relative abundance listed in Table 4). Decision trees were generated by random forest from data in Table 4 with parameter: trees=801, mtry=2.

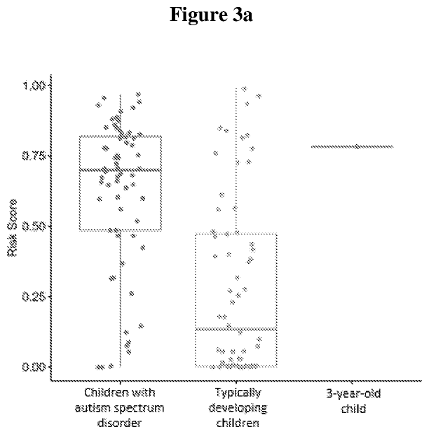

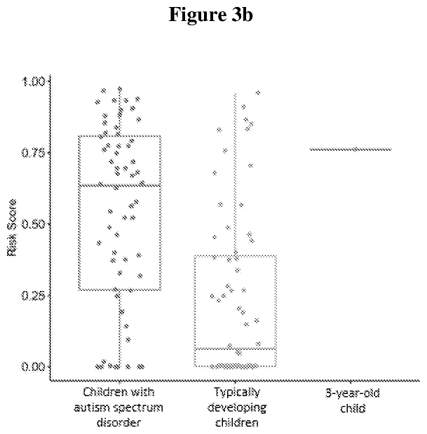

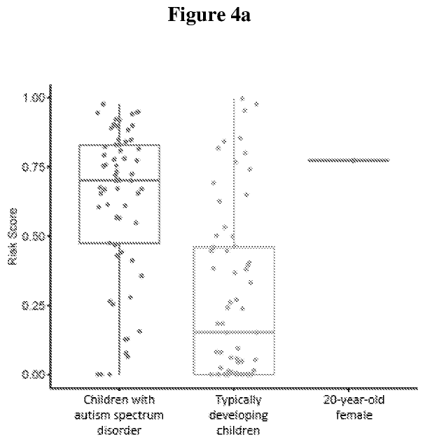

The risk of ASD of a 3-year-old child was determined. The relative abundance of the 5 species listed in Table 3 in fecal sample of this child was determined by metagenomics sequencing and taxonomy assigned as described in method (Table 5). The relative abundances were run down the decision trees and a risk score was generated. The score of the child was 0.78 (FIG. 3a), and therefore the child was deemed to be at risk of ASD.

### Study 2

The relative abundance of Alistipes indistinctus, candidate division TM7 single-cell isolate TM7c, Streptococcus cristatus selected from Table 3 from 64 children with ASD and 64 typically developing children was determined by metagenomics sequencing and taxonomy assigned as described in methods (relative abundance listed in Table 4). Decision trees were generated by random forest from data in Table 4 with parameter: trees=801, mtry=2.

The risk of ASD of a 3-year-old child was determined. The relative abundance of the 3 species above in the fecal sample of this child was determined by metagenomics sequencing and taxonomy assigned as described in method (Table 5). The relative abundances were run down the decision trees and a risk score was generated. The score of the child was 0.833 (FIG. 3b), and therefore the child was deemed to be at risk of ASD.

### Study 3

The relative abundance of 5 species listed in Table 3 from 64 children with ASD and 64 typically developing children was determined by metagenomics sequencing and taxonomy assigned as described in methods (relative abundance listed in Table 4). Decision trees were generated by random forest from data in Table 4 with parameter: trees=801, mtry=2.

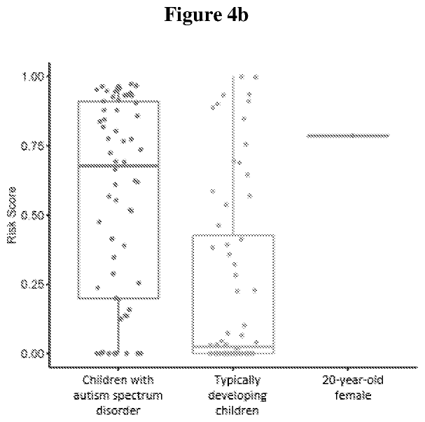

The risk of ASD of a 20-year-old female subject was determined. The relative abundance of the 5 species listed in table 3 in fecal sample of this child was determined by metagenomics sequencing and taxonomy assigned as described in method (Table 6). The relative abundances were run down the decision trees and a risk score was generated. The score of the child was 0.77 (FIG. 4a), and therefore this subject was deemed to be at risk of ASD. This subject was diagnosed with ASD since 2020.

### Study 4

The relative abundance of Alistipes indistinctus, candidate division TM7 single-cell isolate TM7c, Streptococcus cristatus selected from Table 3 from 64 children with ASD and 64 typically developing children was determined by metagenomics sequencing and taxonomy assigned as described in methods (relative abundance listed in Table 4). Decision trees were generated by random forest from data in Table 4 with parameter: trees=801, mtry=2.

The risk of ASD of a 20-year-old female was determined. The relative abundance of the 3 species above in the fecal sample of this child was determined by metagenomics sequencing and taxonomy assigned as described in method (Table 6). The relative abundances were run down the decision trees and a risk score was generated. The score of the child was 0.79 (FIG. 4b), and therefore this subject was deemed to be at risk of ASD.

This subject was diagnosed with ASD since 2020.

TABLE 4
Relative abundance of species listed in Table 3 in 64 children with ASD and 64 typically developing children.

| | Alistipes | candidate division TM7 | Streptococcus | | |
|---|---|---|---|---|---|
| group | indistinctus | single-cell isolate TM7c | cristatus | Eubacterium_{—}limosum | Streptococcus_{—}oligofermentans |
| autism | 0.06343 | 0 | 0 | 0.00138 | 0 |
| autism | 0.0222 | 0 | 0.00222 | 0 | 0 |
| autism | 0.03448 | 0.00044 | 0 | 0.17168 | 0 |
| autism | 0.02463 | 0.00425 | 0 | 0.76747 | 0 |
| autism | 0 | 0 | 0 | 0.04048 | 0 |
| autism | 0.03648 | 0.01191 | 0.00086 | 0 | 0 |
| autism | 0.01712 | 0 | 0 | 0.25384 | 0 |
| autism | 0 | 0.00468 | 5.00E−04 | 0.08619 | 0 |
| autism | 0.00761 | 0 | 0 | 0.00601 | 0 |
| autism | 0 | 0.00319 | 0.00392 | 0.09939 | 0.00056 |
| autism | 0.02715 | 0.00041 | 0.00562 | 0 | 0.00166 |
| autism | 0 | 0.00075 | 0.00043 | 0.00602 | 0.00083 |
| autism | 0 | 0.0017 | 0 | 0.02627 | 0 |
| autism | 0.00027 | 0 | 0.00569 | 0 | 0 |
| autism | 0 | 0 | 0 | 0.14703 | 0 |
| autism | 0.03792 | 0 | 0 | 0.01767 | 0 |
| autism | 0.04835 | 0.00083 | 0 | 1.66373 | 0 |
| autism | 0.00352 | 0 | 0.0011 | 0.03114 | 9.00E−05 |
| autism | 0.02392 | 0.00224 | 0 | 0.05019 | 0 |
| autism | 0.01204 | 0 | 0 | 0 | 0 |
| autism | 0 | 0 | 0 | 0.59767 | 0 |
| autism | 0.10586 | 0 | 0 | 0 | 0 |
| autism | 0.02013 | 0 | 0.00089 | 0 | 0 |
| autism | 0.01058 | 0.00615 | 0 | 0 | 0 |
| autism | 0 | 0 | 0 | 0.38996 | 0 |
| autism | 0 | 0 | 0.00082 | 0.00075 | 0 |
| autism | 0 | 0 | 0.00052 | 0 | 9.00E−05 |
| autism | 0 | 0 | 0 | 0.26536 | 0 |
| autism | 0.04164 | 0 | 0.00063 | 0.02415 | 0.00111 |
| autism | 0 | 0.00599 | 0 | 0.39149 | 0 |
| autism | 0 | 0.01043 | 0.0015 | 0.51814 | 0.0015 |
| autism | 0.00462 | 0.0069 | 0.00183 | 0 | 0 |
| autism | 0.02187 | 0 | 0.00204 | 0.0078 | 0 |
| autism | 0 | 0 | 0 | 0 | 0 |
| autism | 0.00224 | 0 | 0 | 0.55842 | 0 |
| autism | 0 | 0 | 0 | 0 | 0 |
| autism | 0.19464 | 0.0036 | 0 | 0.03229 | 0 |
| autism | 0 | 0.00439 | 0.0015 | 0.0056 | 0 |
| autism | 0.27991 | 0.00645 | 0.00053 | 0.0159 | 0 |
| autism | 0 | 0 | 0 | 0.67253 | 0 |
| autism | 0.00604 | 0 | 0.00028 | 0.0422 | 0 |
| autism | 0 | 0.00146 | 0 | 0 | 0 |
| autism | 0 | 0 | 0.00642 | 0.01552 | 0.00021 |
| autism | 0 | 0 | 0.00074 | 0 | 0 |
| autism | 0.01084 | 0 | 0.00152 | 0.01931 | 0.00473 |
| autism | 0.00441 | 0 | 0 | 0 | 0 |
| autism | 0.11437 | 0.00176 | 0.00095 | 0.03752 | 0 |
| autism | 0 | 0 | 0.00059 | 0 | 0 |
| autism | 0 | 8.00E−05 | 0.00446 | 0 | 0.00044 |
| autism | 0.00377 | 0.00279 | 0.00029 | 0.00122 | 0 |
| autism | 0.35317 | 0.00359 | 4.00E−04 | 0.00217 | 0 |
| autism | 0.02669 | 0.00166 | 0.00178 | 0.23186 | 0 |
| autism | 0 | 0 | 0 | 0 | 0 |
| autism | 0.06026 | 0.00245 | 0.00228 | 0.18623 | 0 |
| autism | 0.09263 | 0 | 0 | 0.04826 | 0 |
| autism | 0 | 0.0015 | 0.00703 | 1.21228 | 8.00E−04 |
| autism | 0.03539 | 0 | 0 | 0.1139 | 0 |
| autism | 0 | 0 | 0.00919 | 0.23437 | 0 |
| autism | 0 | 0 | 0 | 0.30649 | 0 |
| autism | 0.06112 | 0.00128 | 0.00504 | 0 | 0 |
| autism | 0.08314 | 0.00374 | 0.00025 | 0 | 0 |
| autism | 0 | 0 | 0.00733 | 0.48781 | 0 |
| autism | 0.03622 | 5.00E−04 | 0.00621 | 0.00175 | 0.0018 |
| autism | 0.00872 | 0 | 0 | 0.11209 | 0 |
| control | 0 | 0 | 0 | 0 | 0 |
| control | 0 | 0.00214 | 0.00195 | 0.81234 | 8.00E−04 |
| control | 0 | 0 | 0.00505 | 0.00139 | 0 |
| control | 0 | 0 | 0.00119 | 0.00172 | 0 |
| control | 0 | 0 | 0 | 0 | 0 |
| control | 0 | 0 | 0 | 0 | 0 |
| control | 0 | 0 | 0 | 0 | 0 |
| control | 0 | 0 | 0 | 0 | 0 |
| control | 0 | 0 | 0 | 0 | 0 |
| control | 0.00262 | 0 | 0 | 0 | 0 |
| control | 0.01288 | 0 | 0 | 0.10944 | 0 |
| control | 0 | 0 | 0 | 0 | 0 |
| control | 0.01421 | 0 | 0 | 0.01638 | 0 |
| control | 0 | 0 | 0 | 0.01341 | 0 |
| control | 0 | 0 | 0.00266 | 0.00654 | 0 |
| control | 0.00308 | 0 | 0 | 0.01003 | 0 |
| control | 0 | 0 | 0 | 0 | 0 |
| control | 0.00368 | 0.00015 | 0 | 0 | 0 |
| control | 0 | 0 | 0.00028 | 0.02282 | 0 |
| control | 0 | 0 | 0 | 0.05178 | 0 |
| control | 0 | 0 | 0.00172 | 0 | 0 |
| control | 0 | 0 | 0.00036 | 0.00277 | 0 |
| control | 0 | 0.00091 | 0 | 0.57508 | 0 |
| control | 0 | 0.00082 | 0 | 0 | 0 |
| control | 0 | 0.00044 | 0 | 0.23302 | 0 |
| control | 0 | 0.00033 | 0 | 0.02366 | 0 |
| control | 0 | 0 | 0 | 0 | 0 |
| control | 0 | 0 | 0 | 0.01785 | 0 |
| control | 0.00061 | 0 | 0 | 0.00343 | 0 |
| control | 0 | 0.00126 | 0.01262 | 0 | 0 |
| control | 0.05699 | 0 | 0.00049 | 0 | 0 |
| control | 0 | 0 | 0 | 1.39619 | 0 |
| control | 0 | 0.00042 | 0.0081 | 0 | 0.00014 |
| control | 0.00958 | 0 | 0 | 0.00397 | 0 |
| control | 0 | 0 | 0 | 0 | 0 |
| control | 0 | 0 | 0.00186 | 0.03991 | 0 |
| control | 0 | 0 | 0 | 0 | 0 |
| control | 0 | 0 | 0 | 0 | 0 |
| control | 0.00235 | 0 | 0 | 0 | 0 |
| control | 0 | 0 | 0 | 1.13852 | 0 |
| control | 0 | 0 | 0 | 0.01406 | 0 |
| control | 0 | 0 | 0 | 0.01038 | 0 |
| control | 0 | 0 | 0 | 0 | 0 |
| control | 0.00481 | 0 | 0.00085 | 0 | 0 |
| control | 0 | 0 | 0.0018 | 0 | 0 |
| control | 0 | 0 | 0 | 0 | 0 |
| control | 0 | 8.00E−05 | 0 | 0 | 0 |
| control | 0.00038 | 0 | 0 | 0 | 0 |
| control | 0 | 0 | 0 | 0.00044 | 0 |
| control | 0.01036 | 0 | 0 | 0.00926 | 0 |
| control | 0 | 0 | 0 | 0 | 0 |
| control | 0 | 0 | 0 | 0 | 0 |
| control | 0 | 0 | 0.00078 | 0 | 0 |
| control | 0 | 0 | 0 | 0 | 0 |
| control | 0 | 0.00318 | 0 | 0 | 0 |
| control | 0 | 0 | 0.00315 | 0.02503 | 0 |
| control | 0.00717 | 0 | 0 | 0 | 0 |
| control | 0 | 0 | 0 | 0.01518 | 0 |
| control | 0 | 0 | 0 | 0.09747 | 0 |
| control | 0 | 0 | 0 | 0.09511 | 0 |
| control | 0.30825 | 0.00439 | 0 | 0.11324 | 0 |
| control | 0.00866 | 0 | 0 | 0 | 0 |
| control | 0 | 0 | 0 | 0 | 0 |
| control | 0 | 0 | 0 | 0.00635 | 0 |

TABLE 5
Relative abundance of species listed in Table 3 in the 3-year-old child

| | | candidate division | | | |
|---|---|---|---|---|---|
| | Alistipes | TM7 single-cell | Streptococcus | | |
| | indistinctus | isolate TM7c | cristatus | Eubacterium_{—}limosum | Streptococcus_{—}oligofermentans |
| 3-year-old | 0 | 0.00131 | 0.00045 | 0 | 0.00011 |
| child | | | | | |

TABLE 6
Relative abundance of species listed in Table 3 in the 20-year-old female subject

| | | candidate division | | | |
|---|---|---|---|---|---|
| | Alistipes | TM7 single-cell | Streptococcus | | |
| | indistinctus | isolate TM7c | cristatus | Eubacterium_{—}limosum | Streptococcus_{—}oligofermentans |
| 20-year-old | 0 | 0.00337 | 0.00832 | 0 | 0.00193 |
| female subject | | | | | |

### REFERENCES

1 Bolger A M, Lohse M, Usadel B. Trimmomatic: a flexible trimmer for Illumina sequence data. Bioinformatics 2014; 30:2114-20.
2 Truong D T, Franzosa E A, Tickle T L, Scholz M, Weingart G, Pasolli E, et al. MetaPhlAn2 for enhanced metagenomic taxonomic profiling. Nat Methods 2015; 12:902-3.
3 Langmead B, Salzberg S L. Fast gapped-read alignment with Bowtie 2. Nat Methods 2012; 9:357-9.
4 Hadley W, Mara A, Jennifer B, Winston C, Lucy M, Romain F, et al. Welcome to the Tidyverse. Journal of Open Source Software 2019; 4:1686.
5 McMurdie P J, Holmes S. phyloseq: an R package for reproducible interactive analysis and graphics of microbiome census data. PLoS One 2013;8:e61217.
6 Segata N, Izard J, Waldron L, Gevers D, Miropolsky L, Garrett W S, et al. Metagenomic biomarker discovery and explanation. Genome Biol 2011;12:R60.
7 Breiman L. Random Forests. Machine Learning 2001; 45:5-32.
8 Cutler D R, Edwards Jr T C, Beard K H, Cutler A, Hess K T, Gibson J, et al. Random forests for classification in ecology. Ecology 2007; 88:2783-92.
9 Robin X, Turck N, Hainard A, Tiberti N, Lisacek F, Sanchez J C, et al. pROC: an open-source package for R and S+ to analyze and compare ROC curves. BMC Bioinformatics 2011; 12:77.

### Example II: Microbiome-Determined Autism

### Method

### Cohort Description and Study Subjects

An independent validation cohort of ASD (n=8) and typically developing (TD) children (n=10) were recruited to validate the machine learning model described in example 1 (PART I). As in example 1 (PART I), a machine learning model is generated using 5 species listed in Table 3. Briefly, the relative abundance of 5 species listed in Table 3 from 64 children with ASD and 64 TD children was determined by metagenomics sequencing and taxonomy assigned as described in METHODS of PART I (resulting relative abundance are listed in Table 4). Decision trees were generated by random forest from data in Table 4 with parameter: trees=801, mtry=2.

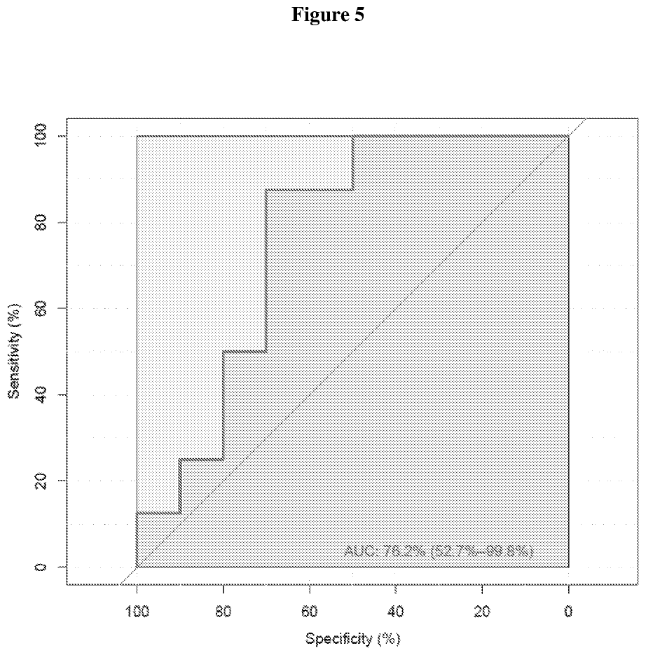

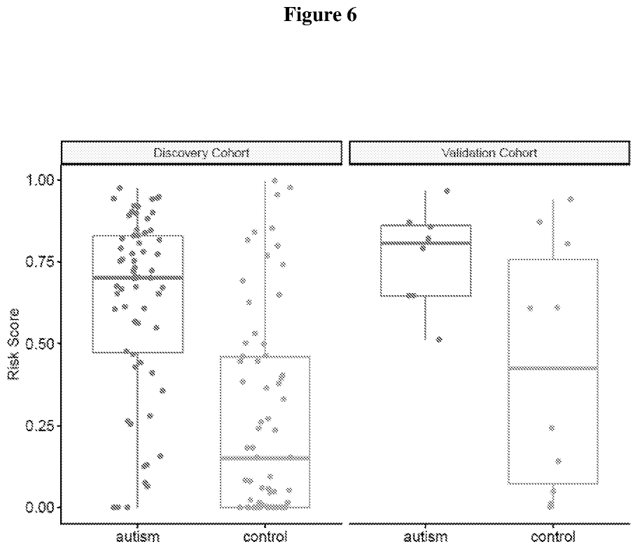

This machine learning model generated from 64 ASD and 64 TD children was used to determine the risk of ASD in each of the 18 children in the validation cohort. The relative abundance of the 5 species in fecal samples from the validation cohort were determined by metagenomics sequencing and taxonomy assigned as described in METHODS of PART I. Resulting relative abundance are listed in Table 7. These relative abundances were run down the decision trees and a risk score was generated. The model showed an AUC of 0.762 in discriminating ASD and TD in the validation cohort (FIG. 5). The average risk score of the ASD and TD children was 0.76 and 0.43, respectively (FIG. 6).

TABLE 7
Relative abundance of species listed in Table 3 in 8 children
with ASD and 10 typically developing children

| | | Candidate division | | | |
|---|---|---|---|---|---|
| | Alistipes | TM7 single-cell | Streptococcus | Eubacterium | Streptococcus |
| group | indistinctus | isolate TM7c | cristatus | limosum | oligofermentans |
| control | 0 | 0.00097 | 0 | 0 | 0 |
| control | 0.08197 | 0 | 0 | 0.04497 | 0 |
| control | 0.00115 | 0 | 0.00275 | 0.00645 | 0 |
| control | 0 | 0 | 0 | 0.27098 | 0 |
| control | 0 | 0 | 0 | 0 | 0 |
| control | 0 | 0.00199 | 0 | 0.00261 | 0 |
| control | 0 | 0.00027 | 0 | 0.00914 | 0 |
| control | 0 | 0 | 0 | 0.02124 | 0 |
| control | 0.08432 | 0 | 0 | 0.01187 | 0 |
| control | 0 | 0.00212 | 0 | 0 | 0 |
| autism | 0 | 0.00966 | 0 | 0 | 0 |
| autism | 0 | 0.00234 | 0 | 0 | 0 |
| autism | 1.39675 | 0 | 0.01058 | 0.11544 | 0 |
| autism | 0 | 0.00611 | 0 | 0 | 0 |
| autism | 0.03593 | 0 | 0 | 0 | 0 |
| autism | 0.13437 | 0 | 0 | 0 | 0 |
| autism | 0.15167 | 0 | 0 | 0.00338 | 0 |
| autism | 0.06872 | 0 | 0 | 0.15475 | 0 |

### Example III: Microbiome-Determined Growth/Development Age

### Methods

### Cohort Description and Study Subjects

A total of 64 typically developing children (aged between 3 and 6 years) were recruited. There were more male (84%) than female. The study was approved by The Joint Chinese University of Hong Kong, New Territories East Cluster Clinical Research Ethics Committee (The Joint CUHK-NTEC CREC, CREC Ref. No: 2016.607). All subjects consented to donate fecal samples and to the questionnaire investigation, where written informed consents were obtained. Fecal samples from the study subjects were stored at −80° C. for downstream microbiome analyses. Children were included as typically developing children without ASD, delays in motor and language development, as well as behaviors as reported by their parents, and those do not have first-degree relatives with ASD.

### Fecal DNA Extraction and DNA Sequencing

Fecal bacterial DNA was extracted by Maxwell® RSC PureFood GMO and Authentication Kit (Promega) with modifications to increase the yield of DNA. Approximately 100 mg from each stool sample was pretreated: stool sample suspended in 1 ml ddH₂O and pelleted by centrifugation at 13,000×g for 1 min. Washed sample was added 800ul TE buffer (PH 7.5), 16ul beta-Mercaptoethanol and 250U lyticase sufficiently mixed and digestion at 37° C. for 90 minutes. Pelleted by centrifugation at 13,000×g for 3 minutes.

After pretreatment, precipitate was re-suspended in 800ul CTAB buffer (Maxwell® RSC PureFood GMO and Authentication Kit following manufacturer's instructions) and mixed well. After samples were heated at 95° C. for 5 minutes and cooled down, nucleic acid was released from the samples by vortexing with 0.5 mm and 0.1 mm beads at 2850 rpm for 15 minutes. Following this, 40ul Proteinase K and 20ul RNase A were added and nucleic acid digested at 70° C. for 10 minutes. Finally, supernatant was obtained after centrifugation at 13,000×g, 5 minutes and placed in a Maxwell® RSC instrument for DNA extraction. The extracted fecal DNA was used for ultra-deep metagenomics sequencing via Ilumina Novaseq 6000 (Novogene, Beijing, China).

### Quality Control of Raw Sequences

Raw sequence reads were trimmed by Trimmomatic¹(v0.38) firstly and then separation of non-human reads from contaminant host reads. There were some steps to acquire clean reads: 1) Remove adapters; 2) Scan the read with a 4-base wide sliding window, removing reads when the average quality per base drop below 20; 3) Drop reads below the 50 bases long. Trimmed sequence reads were mapped to human genome (Reference database: GRCh38 p12) by KneadData (v0.7.2) to remove reads originated from the host. Pair-end two reads were concatenated together.

### Analysis of the Bacterial Microbiome

Profiling of the composition of bacterial communities was performed on metagenomic trimmed reads via MetaPhlAn2 (v2.7.5)². Mapping reads to clade-specific markers gene and annotation of species pangenomes was done through Bowtie2 (v2.3.4.3)³. The output table contained bacterial species and its relative abundance in different levels, from kingdom to strain level. Bacterium species and chronological age correlations performed by Spearman's correlation analysis was conducted via psych package (1.9.12.31) in R.

### Machine Learning Model

Random forest (RF) was chosen to build microbiota age prediction model using fecal microbes from 64 typically developing children because of its superior performance for mean prediction with learning method for regression. Random Forest⁷ is one of the most popular approaches in metagenomic data analysis to identify the discriminative features and build prediction models. As a widely used ensemble learning algorithm, Random Forest consists of a series of classification and regression trees (CARTs) to form a strong mean prediction. A subset of data randomly sampled from the original dataset with replacement is known as bootstrap sampling, applying to build the trees. When the training dataset for the current tree is drawn by the model votes or averaging, into a single ensemble model that ends up outperforming any individual decision tree's output. Bootstrap method,

$$1 - {( 1 - \frac{1}{N} )}^{N}$$

observations are left out from the overall dataset. With infinite N, there are 36.8% data not occurred in the training samples called out-of-bag (OOB) observations, which would not be used for constructing the trees. In addition, extra randomness was introduced to the random forest as each decision tree splits nodes based on a random subset of features selected from the overall features. The features with the higher % IncMSE (Increased in mean squared error (%)) represent features that have greater contribution in prediction model. With different subsets of data and features, the algorithm is able to train different trees and obtain the final result by averaging the result from the tree models. In addition to the prediction model, Random Forest has the capability to assess the importance of variables⁸. To obtain a single prediction for a single OOB observation, these predicted responses can be averaged. To measure the importance of a given variable, the values of the variable in the OOB data are randomly altered, and then the changed OOB data is used to generate new predictions. The difference of the error rate between the altered and the original OOB observations divided by the standard error is calculated as % IncMSE (estimated with out-of-bag) the importance of a variable. To predict a new sample, the features of the sample were passed down to each tree to estimate the average value. The Random Forest used the average probability of all trees to determine the final result.

A total 64 typically developing children were included as the discovery cohort for modeling. The importance value of each species to the regression model was evaluated by recursive feature elimination. According to descending importance value, top 5 bacterial taxa were selected to build model. These analysis was done using R packages randomForest v4.6-14⁷.

### Results

Gut bacterial species correlated with chronological age in typically developing children

To assess the correlation between bacterial species and children chronological age, Spearman's correlation coefficient between the 2 factors was calculated. Statistical significance was determined for all pairwise comparisons. There are both positive (relative abundance increase with age) and negative correlations (relative abundance decrease with age). Only statistically significant correlations with absolute coefficient>0.2 were shown in the table below. For example, the species Bacteroides thetaiotaomicron was significantly increased with children's age. Bacteroides thetaiotaomicron can help children metabolizing a diverse range of polysaccharides when they have abundant carbohydrate-rich diets.

TABLE 8
Bacterial Species significantly correlated
with children chronological age

| | | | Mean relative |
|---|---|---|---|
| | | | abundance |
| | Spearman's | | in typically |
| | correlation | | developing |
| Species | coefficient | NCBI: txid | children (%) |
| Enterococcus avium | −0.29 | 33945 | 0.04 |
| Streptococcus vestibularis | −0.28 | 1343 | 0.01 |
| Anaerostipes unclassified | −0.27 | — | 0.19 |
| Streptococcus gordonii | −0.27 | 1302 | 0.00 |
| Blautia hansenii | −0.24 | 1322 | 0.00 |
| Clostridium hathewayi | −0.22 | 154046 | 0.16 |
| Parabacteroides | 0.20 | — | 0.16 |
| unclassified | | | |
| Ruminococcus albus | 0.20 | 1264 | 0.00 |
| Adlercreutzia equolifaciens | 0.20 | 446660 | 0.11 |
| Eubacterium_sp_3_1_31 | 0.20 | 457402 | 0.28 |
| Mitsuokella multacida | 0.21 | 52226 | 0.03 |
| Alistipes onderdonkii | 0.21 | 328813 | 0.05 |
| Bacteroides fragilis | 0.21 | 817 | 0.45 |
| Lachnospiraceae | 0.21 | 665950 | 0.05 |
| bacterium_3_1_46FAA | | | |
| Roseburia intestinalis | 0.21 | 166486 | 2.59 |
| Bacteroides uniformis | 0.21 | 820 | 0.78 |
| Eubacterium brachy | 0.23 | 35517 | 0.00 |
| Bacteroides | 0.25 | 818 | 0.44 |
| thetaiotaomicron | | | |
| Dorea formicigenerans | 0.27 | 39486 | 0.62 |
| Bilophila unclassified | 0.28 | — | 0.11 |
| Bacteroides xylanisolvens | 0.28 | 371601 | 0.05 |
| Faecalibacterium | 0.29 | 853 | 9.27 |
| prausnitzii | | | |

As such, bacteria listed in Table 8 can be used in different combinations to build an assessment model to determine the age of growth and development in a child and whether microbiome restoration therapy or supplementation is required. The relative abundance can be determined using as a panel of qPCR primer or by metagenomics sequencing to determine the development of gut microbiota.

Furthermore, bacteria listed in Table 8 that have a positive correlation coefficient (Spearman's correlation coefficient) may be supplemented to children to support growth and development in children. The relative abundance should increase to a level higher than or equal to the mean relative abundance of typically developing children listed in Table 8.

### Determination of Age of Growth and Development Using Machine Learning Model

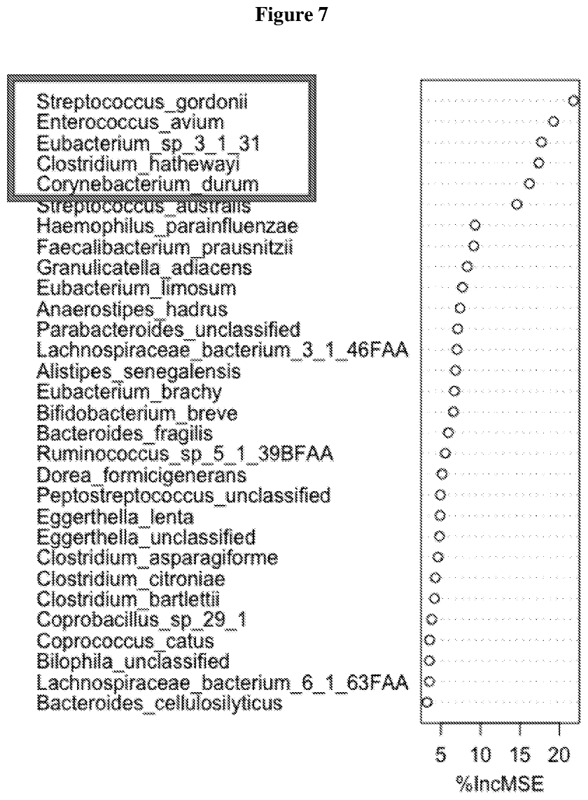

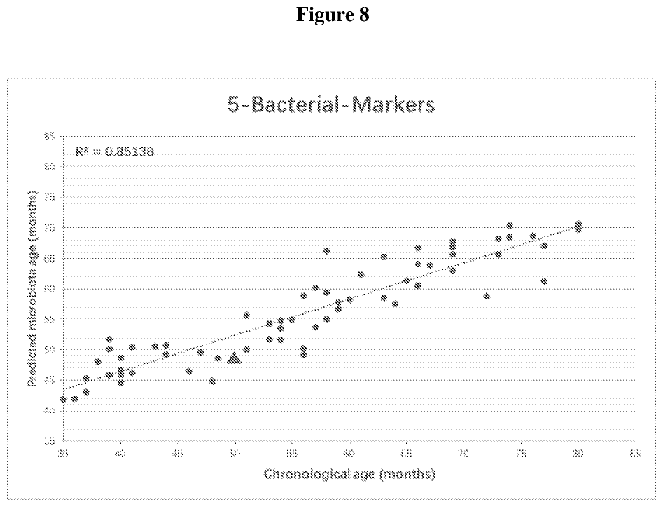

With regression random forest analysis, it was discovered that the species Streptococcus gordonii, Enterococcus avium, Eubacterium_sp_3_1_31, Clostridium hathewayi, Corynebacterium durum (FIG. 7, Table 9) were top 5 most important predictor variable in typically developing children. 5 bacterial taxa were used to build a machine learning model to predict microbiota age. % IncMSE is computed from data. Importance of variables used in random forest modeling which indicates the increase of the Mean Squared Error when given variable is randomly permuted. The final models using these 5 markers has an r-squared (0.85) (FIG. 8).

TABLE 9
Bacterial Species included in the Machine Learning Model
for Determination of Age of Growth and Development

| Bacterial Species | NCBI: txid |
|---|---|
| Streptococcus gordonii | 1302 |
| Enterococcus avium | 33945 |
| Eubacterium sp. 3_1_31 | 457402 |
| Clostridium hathewayi | 154046 |
| Corynebacterium durum | 61592 |

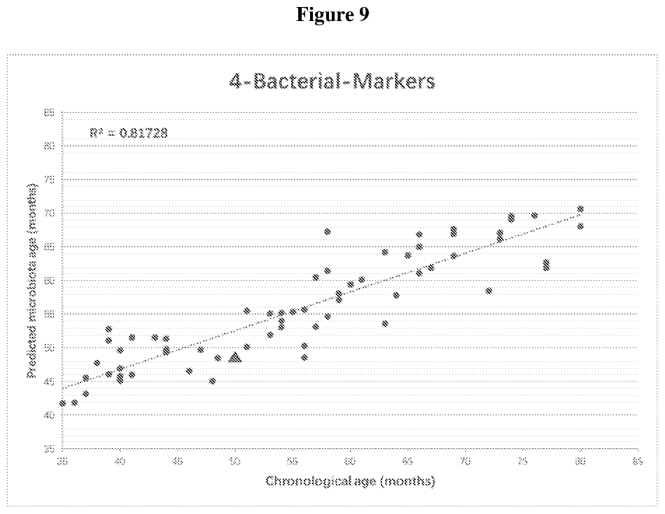

Thus, to determine the risk of growth and developmental delay in a child, the following steps will be carried out:
1. Obtain a set of training data by determining the relative abundance of species selected from Table 9* in a cohort of typically developing children.
2. Determine the relative abundance of these species in the child whose risk of growth and developmental delay is to be determined.
3. Compare the relative abundance of these species in the subject with the training data using random forest model.
4. Decision trees will be generated by random forest from the training data. The relative abundances will be run down the decision trees and generate a mean prediction, which correspond to the predicted age of growth and development. If the predicted age of growth and development is lower than the chronological age of the child, the child is a risk of growth and developmental delay.
   *species selected from Table 9 may comprise of
   1. Five bacterial species (Streptococcus gordonii, Enterococcus avium, Fubacterium_sp_3_1_31, Clostridium hathewayi, Corynebacterium durum; FIG. 8),
   2. Four species (Streptococcus gordonii, Enterococcus avium, Eubacterium_sp_3_1_31, Clostridium hathewayi; FIG. 9),
   3. Three species (Streptococcus gordonii, Enterococcus avium, Eubacterium_sp_3_1_31;

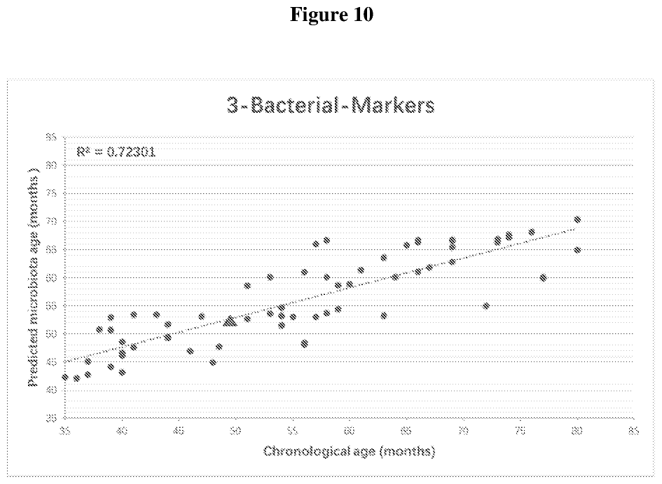

FIG. 10),

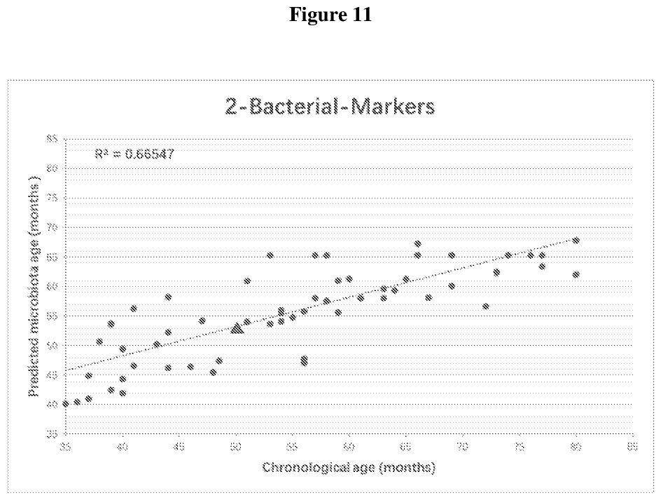

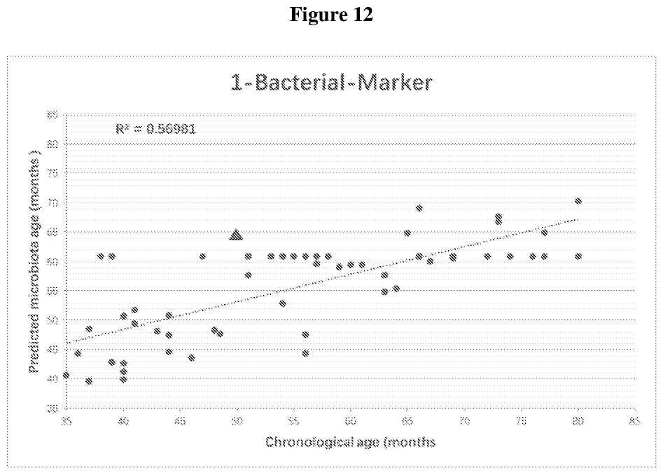

4. Two species (Streptococcus gordonii and Enterococcus avium; FIG. 11), or
5. One species (Streptococcus gordonii; FIG. 12).

### Study 1

Five bacterial species (Streptococcus gordonii, Enterococcus avium, Fubacterium_sp_3_1_31, Clostridium hathewayi, Corynebacterium durum) listed in Table 9 from 64 typically developing children was determined by metagenomics sequencing and taxonomy assigned as described in methods (relative abundance listed in Table 10). Decision trees were generated by random forest from data in Table 10 with parameter: ntree=10000, proximity=TRUE, importance=TRUE, nPerm=10.

The risk of growth and developmental delay of a 3-year-old child was determined. The relative abundance of the 5 species above in fecal sample of this child was determined by metagenomics sequencing and taxonomy assigned as described in method. The relative abundances were run down the decision trees and a predicted age of growth and development was generated. The predicted age of this child was 48.3 month (FIG. 8). The child is deemed to have a low risk of growth and developmental delay.

### Study 2

Four species (Streptococcus gordonii, Enterococcus avium, Eubacterium_sp_3_1_31, Clostridium hathewayi) listed in Table 9 from 64 typically developing children was determined by metagenomics sequencing and taxonomy assigned as described in methods (relative abundance listed in Table 10). Decision trees were generated by random forest from data in Table 10 with parameter: ntree=10000, proximity=TRUE, importance=TRUE, nPerm=10.

The risk of growth and developmental delay of a 3-year-old child was determined. The relative abundance of the 4 species above in fecal sample of this child was determined by metagenomics sequencing and taxonomy assigned as described in method. The relative abundances were run down the decision trees and a predicted age of growth and development was generated. The predicted microbiota age in child was 48.3 month (FIG. 9). The child is deemed to have a low risk of growth and developmental delay.

### Study 3

Three species (Streptococcus gordonii, Enterococcus avium, Eubacterium_sp_3_1_31) listed in Table 9 from 64 typically developing children was determined by metagenomics sequencing and taxonomy assigned as described in methods (relative abundance listed in Table 10). Decision trees were generated by random forest from data in Table 10 with parameter: ntree=10000, proximity=TRUE, importance=TRUE, nPerm=10.

The risk of growth and developmental delay of a 3-year-old child was determined. The relative abundance of the 3 species above in the fecal sample of this child was determined by metagenomics sequencing and taxonomy assigned as described in method. The relative abundances were run down the decision trees and a predicted age of growth and development was generated. The predicted age of this child was 52.6 month (FIG. 10). The child is deemed to have a low risk of growth and developmental delay.

### Study 4

The relative abundance of Streptococcus gordonii and Enterococcus avium in 64 typically developing children was determined by metagenomics sequencing and taxonomy assigned as described in methods (relative abundance listed in Table 10). Decision trees were generated by random forest from data in Table 10 with parameter: ntree=10000, proximity=TRUE, importance=TRUE, nPerm=10.

The risk of growth and developmental delay of a 3-year-old child was determined. The relative abundance of the 2 species above in the fecal sample of this child was determined by metagenomics sequencing and taxonomy assigned as described in method. The relative abundances were run down the decision trees and a predicted age of growth and development was generated. The predicted age of this child was 53.2 month (FIG. 11). The child is deemed to have a low risk of growth and developmental delay.

### Study 5

The relative abundance of Streptococcus gordonii in 64 typically developing children was determined by metagenomics sequencing and taxonomy assigned as described in methods (relative abundance listed in Table 10). Decision trees were generated by random forest from data in Table 10 with parameter: ntree=10000, proximity=TRUE, importance=TRUE, nPerm=10.

The risk of growth and developmental delay of a 3-year-old child was determined. The relative abundance of the one species above in the fecal sample of this child was determined by metagenomics sequencing and taxonomy assigned as described in method. The relative abundances were run down the decision trees and a predicted age of growth and development was generated. The predicted age of this child 64.9 month (FIG. 12). The child is deemed to have a low risk of growth and developmental delay.

TABLE 10
Relative abundance of species listed in Table 9 in 64 typically developing children

| | Streptococcs | Enterococcus | Eubacterium | Clostridium | Corynebacterium |
|---|---|---|---|---|---|
| group | gordonii | avium | sp. 3_1_31 | hathewayi | durum |
| control | 0.00025 | 0 | 0 | 0.05232 | 0.0031 |
| control | 0.00645 | 1.19603 | 0 | 0.31152 | 0.00291 |
| control | 0.00566 | 0 | 0.06364 | 0.005 | 0.0022 |
| control | 0.00076 | 0 | 0 | 0.03461 | 0.00472 |
| control | 0 | 0.02123 | 1.71828 | 0.4845 | 0.00319 |
| control | 0.00341 | 0 | 0.00209 | 0.00525 | 0.001 |
| control | 0 | 0 | 0.10125 | 0.024 31 | 0.01712 |
| control | 0 | 0.1611 | 0 | 0.18187 | 0.00315 |
| control | 0 | 0 | 0.00876 | 0.02984 | 0.00085 |
| control | 0 | 0 | 0.01711 | 0.02107 | 0.00138 |
| control | 0.00792 | 0.01802 | 0.647 | 0.21328 | 0.00102 |
| control | 0.00275 | 0 | 0.13588 | 0.00914 | 0.02866 |
| control | 0.00582 | 0.00078 | 0.08747 | 0.37794 | 0.00132 |
| control | 0.00228 | 0 | 0 | 0.03605 | 0.00534 |
| control | 0 | 0 | 0 | 0.07575 | 0.00999 |
| control | 0.00408 | 0 | 0 | 0.2883 | 0.00783 |
| control | 0.00061 | 0 | 0 | 0 | 0.00306 |
| control | 0 | 0 | 0.04373 | 0.00532 | 0.00659 |
| control | 0 | 0 | 0.00309 | 0.12021 | 0 |
| control | 0 | 0 | 0.44467 | 0.03723 | 0.00738 |
| control | 0 | 0.00456 | 0 | 0.03728 | 0.00347 |
| control | 0.00095 | 0 | 0 | 0.06264 | 0.00507 |
| control | 0.00686 | 0.01027 | 1.35995 | 0.24678 | 0.00403 |
| control | 0.01335 | 0.00444 | 0 | 0.00296 | 0.00426 |
| control | 0.00269 | 0.00033 | 0.27761 | 0.06356 | 0.0058 |
| control | 0.013 | 7.00E−05 | 0.00499 | 0.46143 | 0.00379 |
| control | 0.00119 | 0 | 0 | 0.12822 | 0 |
| control | 0 | 0 | 0.0025 | 0.0366 | 0 |
| control | 9.00E−05 | 0 | 0.0168 | 0.01287 | 0.00322 |
| control | 0.00131 | 0.05037 | 0 | 0.14731 | 0.0034 |
| control | 0.02961 | 0 | 0 | 0.02263 | 0.00332 |
| control | 0.0046 | 0.00152 | 0.0031 | 0.19191 | 0 |
| control | 0.00224 | 0.05093 | 0 | 0.12189 | 0.00316 |
| control | 0 | 0 | 0.15276 | 0.03083 | 0.00191 |
| control | 0.00539 | 0 | 0.01782 | 0.08201 | 0 |
| control | 0 | 0 | 0 | 1.49382 | 0 |
| control | 0.00123 | 0 | 0 | 0.4475 | 0 |
| control | 0.01334 | 0 | 0 | 0.16888 | 0 |
| control | 0 | 0 | 0.02795 | 0.01196 | 0.00047 |
| control | 0.00638 | 0.00058 | 0 | 0.5053 | 0 |
| control | 0 | 0 | 0 | 0.07495 | 0.00072 |
| control | 0 | 0.01643 | 0.59712 | 0.10561 | 0.00039 |
| control | 0 | 0.07846 | 0 | 0.09432 | 0 |
| control | 0 | 0.0037 | 4.84989 | 0.02214 | 0.01237 |
| control | 0 | 0.00126 | 5.06255 | 0.27765 | 0.01787 |
| control | 0 | 0.01167 | 0 | 0.50354 | 0 |
| control | 0.00961 | 0.17927 | 0 | 0.43863 | 0.00589 |
| control | 0 | 0.00057 | 0 | 0.02217 | 0 |
| control | 0.0181 | 0 | 0.02612 | 0.0031 | 0.00949 |
| control | 0.00987 | 0 | 0.37383 | 0.18229 | 0 |
| control | 0 | 0.00715 | 0 | 0.04786 | 0.00066 |
| control | 0 | 0.00014 | 0 | 0.04506 | 0.00049 |
| control | 0.00637 | 0.10742 | 0 | 0.79462 | 0.00606 |
| control | 0.00601 | 0 | 0 | 0.01005 | 0.00131 |
| control | 0.01657 | 0 | 0.03189 | 0.10284 | 0.00776 |
| control | 0.0047 | 0 | 0 | 0.00644 | 0 |
| control | 0 | 0.04207 | 0 | 0.24163 | 0 |
| control | 0.00645 | 0.00078 | 0 | 0.07611 | 0.00406 |
| control | 0.00655 | 0.01906 | 1.48991 | 0.05491 | 0.00473 |
| control | 0.0069 | 0 | 0 | 0.06296 | 0.00094 |
| control | 0 | 0 | 0.07597 | 0.15436 | 0.00195 |
| control | 0.00554 | 0 | 0.01795 | 0.02848 | 0.00841 |
| control | 0.00084 | 0.3651 | 0 | 0.08301 | 0.00159 |
| control | 0.00755 | 0 | 0.00046 | 0.00387 | 0 |

TABLE 11
Relative abundance of species listed in Table 9 in the 3-year-old child

| | Species | | | | |
|---|---|---|---|---|---|
| | Streptococcus | Enterococcus | Eubacterium | Clostridium | Corynebacterium |
| | gordonii | avium | sp. 3_1_31 | hathewayi | durum |
| 3-year-old | 0.00119 | 0.09925 | 0 | 1.51249 | 0.0042 |
| child | | | | | |

### ASD and Age had the Most Significant Impact on Children's Gut Microbiome

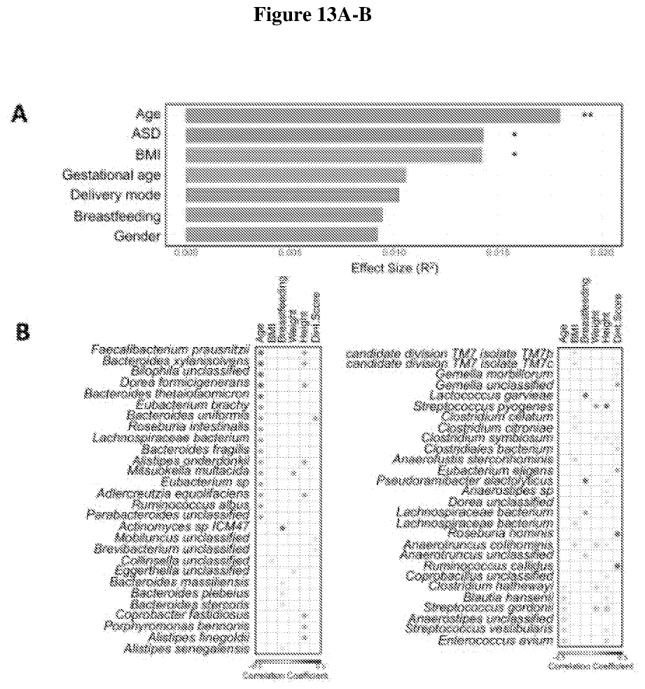

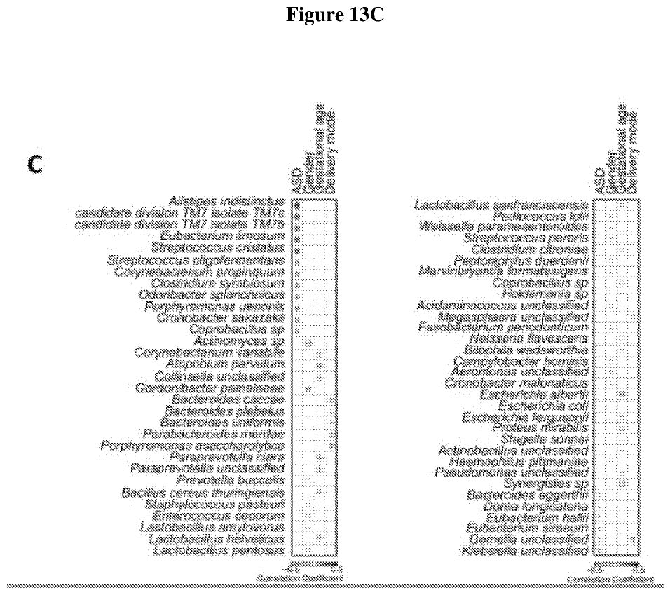

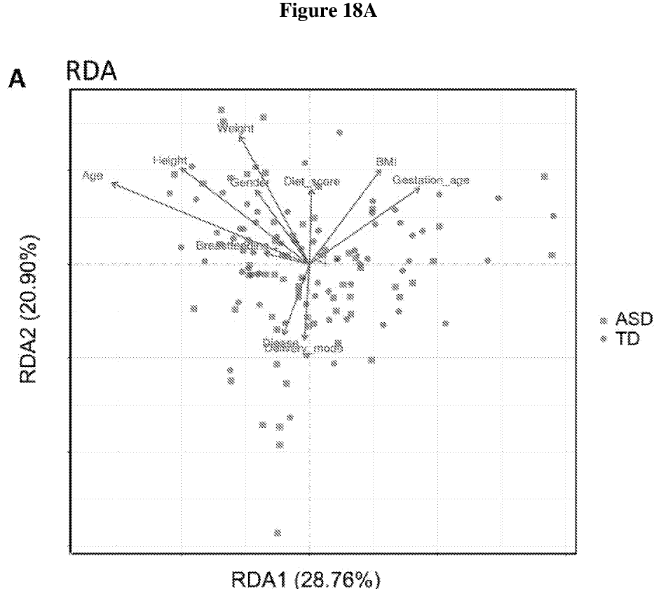

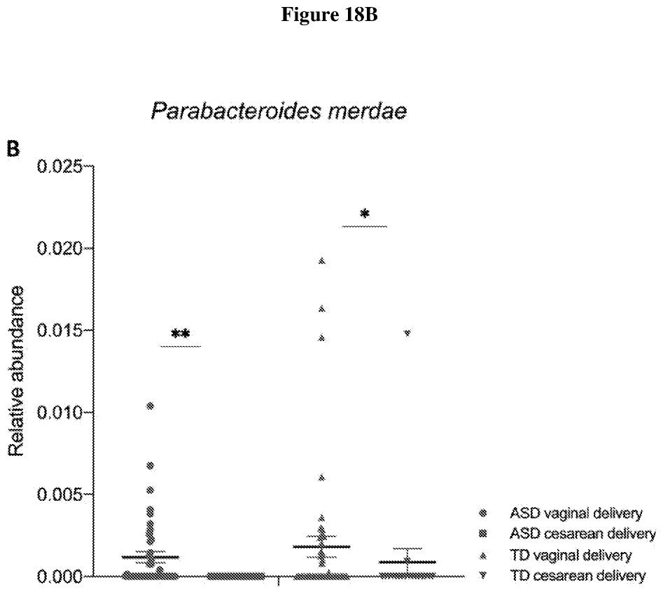

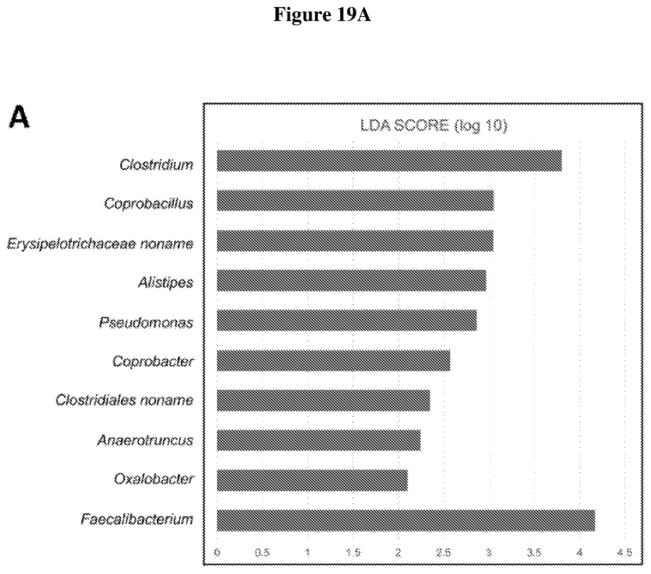

Chinese children aged 3 to 6 years in Hong Kong (64 ASD versus 64 TD children) were enrolled and examined the effects of host factors on configuration of children's fecal microbiome. Among host factors examined, ASD, chronological age and Body Mass Index (BMI) showed the most significant impact on the fecal microbiome ranked according to effect size (FIG. 13A, PERMANOVA). The impact of ASD and age on altered gut microbiome are individually independent of other host factors (FIG. 18A). To further explore how host factors impacted microbiome composition, the correlations between individual host factors and the bacterial species were interrogated. One hundred and eleven bacterial species were identified to significantly correlated with ASD, chronological age, BMI, duration of breastfeeding, weight, height, diet score (food frequency), gender, gestational age and delivery mode (all FDR adjusted P value<0.05, Spearman's and Kendall's correlation value>0.2 or <−0.2, FIG. 13B-C). Abundance of the species, Faecalibacterium prausnitzii and Bacteroides xylanisolvens were positively associated with children's chronological age while the species, Enterococcus avium, Streptococcus gordonii, and Streptococcus vestibularis negatively correlated with children chronological age (FIG. 13B). The species Alistipes indistinctus, candidate TM7b, TM7c, Eubacterium limosum and Streptococcus cristatus were positively correlated with ASD (abundance significantly higher in ASD versus TD children, FIG. 13C). Children delivered via cesarean section showed correlation with Parabacteroides merdae, which were significantly decreased in children delivered via cesarean section compared with those birthed by vaginal delivery and the taxa reduced in ASD in both two delivery modes (FIG. 18B). Altogether, these data indicate that host factors have prominent impacts in shaping children's gut microbiota; among them, ASD, chronological age, BMI had the strong effect size in microbiome variation.

### Identification of Faecal Bacteria Species as Potential Biomarker for ASD

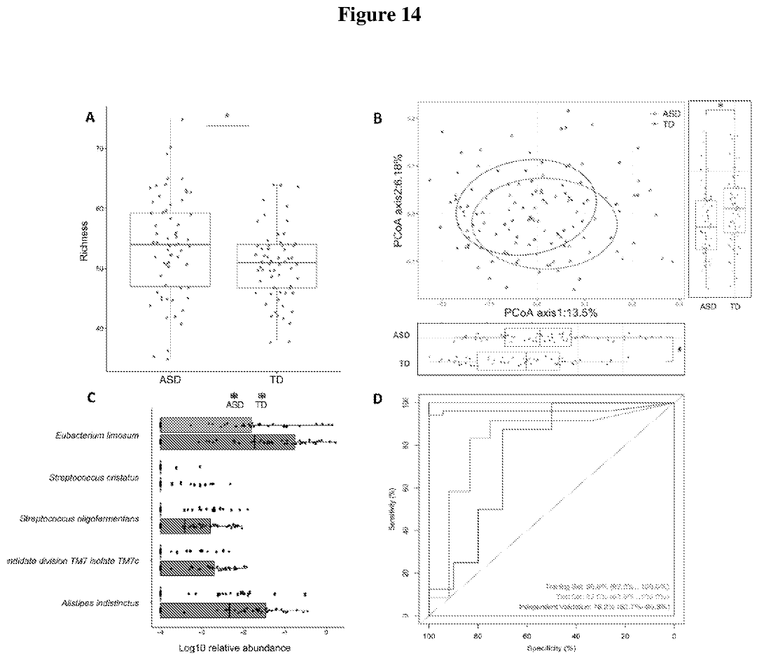

The gut microbiome composition in children with ASD was altered at multiple taxonomic levels compared with TD children. Microbiome richness was higher in children with ASD than age- and BMI-matched TD children (BMI: 15.31±1.87 versus 15.38±1.42 respectively) (t-test, p-value=0.021, FIG. 14A). Within group individual variation of bacterial richness was also higher in children with ASD (54.0[47.0-59.3] versus 51.0[46.8-54.0] in ASD versus TD respectively). At the composition level, the gut microbiome structure in children with ASD and TD children were significantly different, as demonstrated by different clustering and separation in the principal coordinate analysis (PCoA) plot (FIG. 14B, t-test, p=0.0390 and 0.0136, based on the Bray-Curtis dissimilarities presented on both of the PCoA axes). In addition, the gut microbiomes of children with ASD were more heterogeneous than TD children (FIG. 14B).

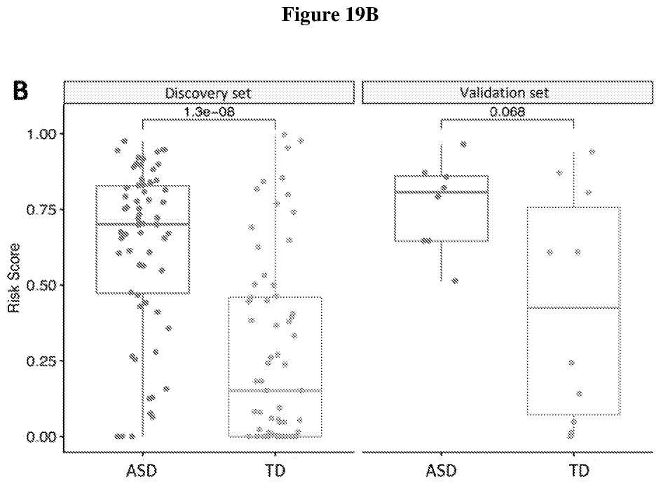

At the genus level, Clostridium and Coprobacillus were largely enriched in children with ASD (FIG. 19A, Kruskal-Wallis test, p-values 0.032 and 0.022 respectively), whereas Faecalibacterium, known to produce butyrate (Machiels et al., Gut 63(8): 1275-1283, 2014), was significantly decreased in children with ASD compared with TD children (FIG. 19A, Kruskal-Wallis test, p-value=0.013). At the species level, five bacterial species differed between children with ASD and TD children, including Alistipes indistinctus, candidate division_TM7_isolate_TM7c, Streptococcus cristatus, Eubacterium limosum and Streptococcus oligofermentans (identified by Random Forest via 10-fold cross-validation, FIG. 14C). Based on the five potential species markers, a random forest model showed an area under curve (AUC) value of 80.3% in distinguishing between ASD and TD in a discovery set. In independent validation set (10 TD children and 8 children with ASD), the same model achieved 76.2% in AUC (FIG. 14D). The possibility of randomly generated decision trees that predicted children to have ASD was increased in ASD versus TD (FIG. 19B).

Gut Bacterium-Bacterium Ecological Network is Impaired in Children with ASD

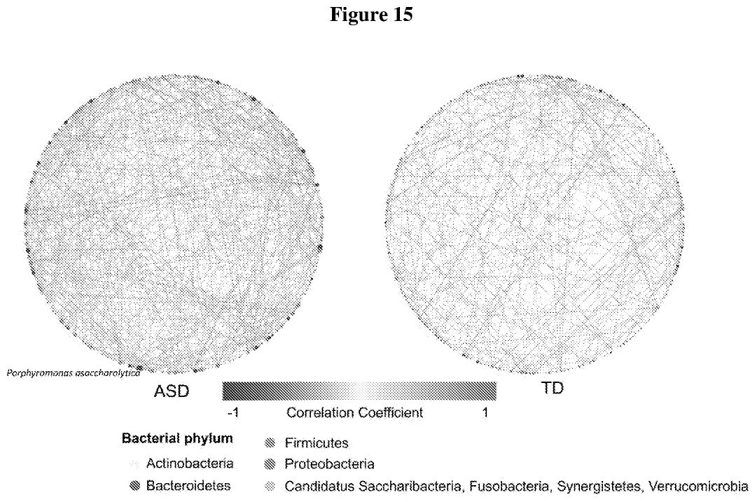

The ecological interaction of bacterium-bacterium in the gut of ASD and TD group was next assessed by evaluating spearman's correlation between bacterial species. Majority of bacterium-bacterium correlations in both ASD and TD children were positive correlations (FIG. 15). A stronger correlation network was observed in ASD group in contrast to the sparse correlation network in TD children, as indicated by both the number (671 versus 368) and correlation coefficients of significant bacterium-bacterium correlations were higher in the gut microbial community of ASD than TD (FIG. 15, p-value<0.05, |correlation coefficient|>0.5). In TD children, bacteria from the phylum Firmicutes showed most inter-species interactions, whereas species from the phylum Bacteroidetes showed robust bacterial-bacterial correlations in the ecological network of ASD (FIG. 15). In particular, the correlations of Porphyromonas asaccharolytica, which serve as opportunistic pathogens, were intensive in ASD children. Such changes in the gut microbiome ecological network indicates that inter-species communication/interplay was significantly altered in ASD children's gut.

Pathways Related to Neurotransmitter Biosynthesis were Decreased in the Gut Microbiome of ASD

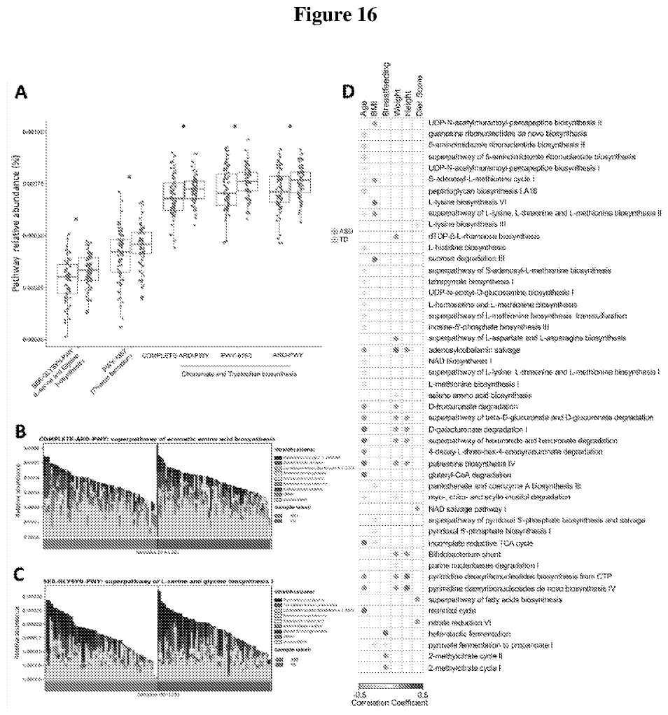

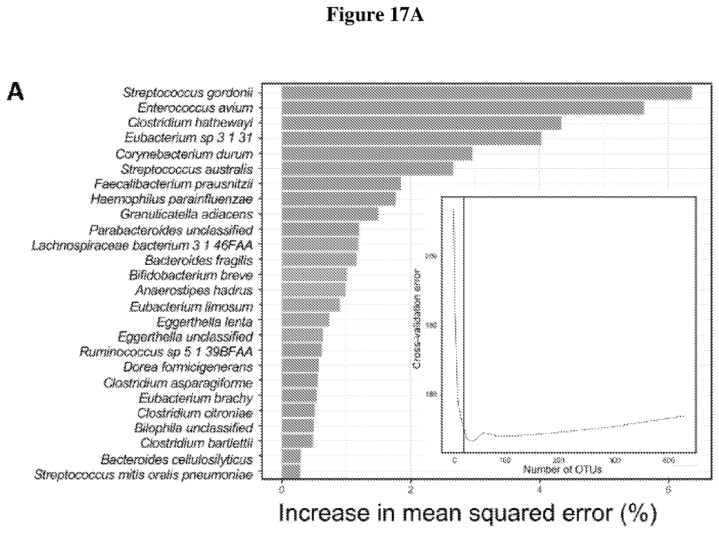

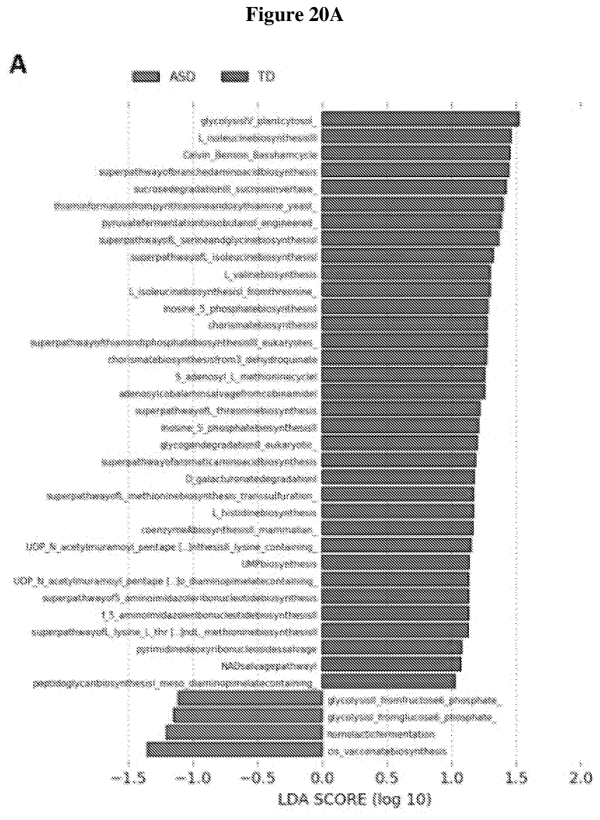

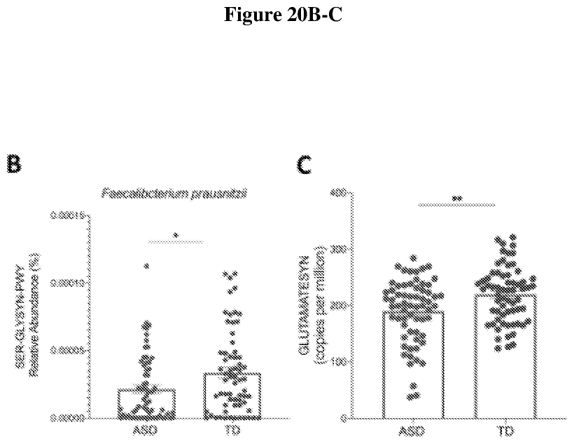

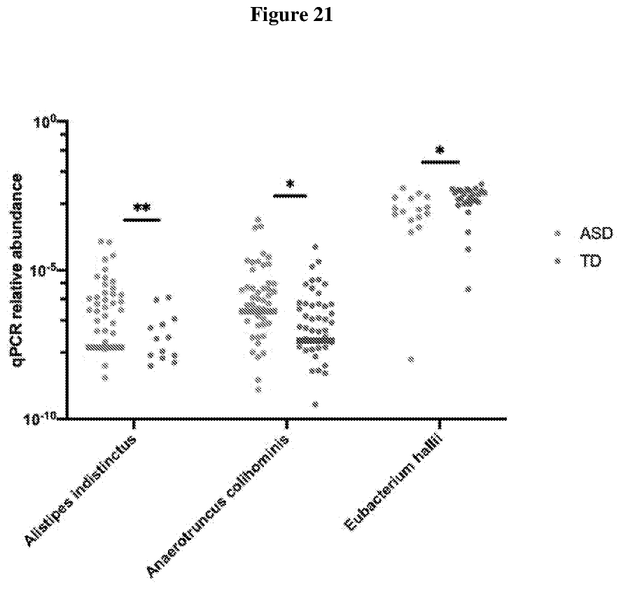

To understand alterations in gut microbiome functions in relation to compositional changes in ASD, the genetic abundance of constituent functional modules (gene families) was profiled using HUMAnN2 (Franzosa et al., Nature methods 15(11): 962-968, 2018). Pathways for essential amino acid biosynthesis (L-threonine, L-isoleucine, L-leucine, L-valine), glucose metabolism, nucleotide biosynthetic and vitamin B biosynthetic were significantly decreased in ASD children (FIG. 20A). Importantly, among them, pathways related to neurotransmitter biosynthesis were decreased in the gut microbiome of ASD compared to TD (FIG. 16A). The pathways, ARO-PWY and PWY-6163, involving in biosynthesis of chorismate (a precursor for tryptophan biosynthesis), were significantly decreased in the gut microbiome of ASD group compared to TD group (FDR adjusted p values<0.05, FIG. 16A). Concomitantly, the COMPLETE-ARO-PWY function corresponding to the biosynthesis of aromatic amino acids (including L-tryptophan, L-phenylalanine, L-tyrosine), all starting from the principal common precursor chorismate(Pittard and Yang, EcoSal Plus 3(1), 2008), was also decreased in ASD (FIG. 16A). Tryptophan is a key precursor for the metabolites kynurenine and serotonin, both of them were critical neurotransmitters implicated in counteracting depression and other psychiatric disorders (Vaswani et al., Progress in neuro-psychopharmacology and biological psychiatry 27(1): 85-102, 2003). In addition, pathway of glycine (inhibitory neurotransmitter) biosynthesis was depleted in the fecal microbiome of children with ASD (FIG. 16A).

Collectively, neurotransmitters enable signal transmission across synapses to nerve cells, where synaptic dysfunction is thought to crucially contribute to the pathophysiology of ASD (Zoghbi and Bear (2012), “Synaptic dysfunction in neurodevelopmental disorders associated with autism and intellectual disabilities.” Cold Spring Harbor perspectives in biology 4(3): a009886). As such, changes in these pathways, particularly for tryptophan and glycine anabolism/metabolism, in the functionality of ASD microbiome could lead to abnormal neurotransmitter synthesis and therefore relays to the host. The species Ruminococcus sp. 5_1_39BFAA, Eubacterium rectale and Ruminococcus bromii, Faecalibacterium prausnitzii were dominant contributors to the biosynthesis of L-tryptophan and glycine respectively (FIG. 16B-C). Notably, contribution of F. prausnitzii in serine-glycine metabolism pathway was significantly decreased in children with ASD compared to TD children (FIG. 20B). Altogether, these data show that the microbiome functionalities associated with neurotransmitter synthesis were markedly reduced in ASD children, which may have profound functional consequence for the psychiatric abnormalities in ASD.

Beyond that, the abundance of microbial genes encoding for glutamate synthase was also significantly decreased in ASD children compared to TD children (FIG. 20C). Glutamate synthase is an enzyme that manufactures glutamate which is the most abundant excitatory neurotransmitter in the vertebrate nervous system (Zhou and Danbolt, Journal of neural transmission 121(8): 799-817, 2014). The disarrangement in the abundance of glutamate synthase-coding genes may have detrimental effect on host psychiatric response.

Building upon the gut microbiome functionality profile and clinical parameters of the study subjects, the inventors explored the relationship between the abundance of microbiome functional modules and host factors, via correlation analysis. It was found that age had the most profound effect in shaping the functionality of the gut microbiome in children, as demonstrated by the most abundant associations between children's age, among the examined host factors, and abundances of microbial functional modules (30 significant associations, P value<0.05, Spearman's correlation value>0.2 or <−0.2, FIG. 16D). Of those associations, energy metabolism-related microbial functional pathways (sugar degradation; carboxylate degradation) were increased with age, whereas purine nucleotide biosynthesis pathways were decreased with age (FIG. 16D). Collectively, chronological age had an overall large effect on the functionality of the gut microbiome in children.

### Distortions in the Development of Growth-Associated Bacteria in ASD

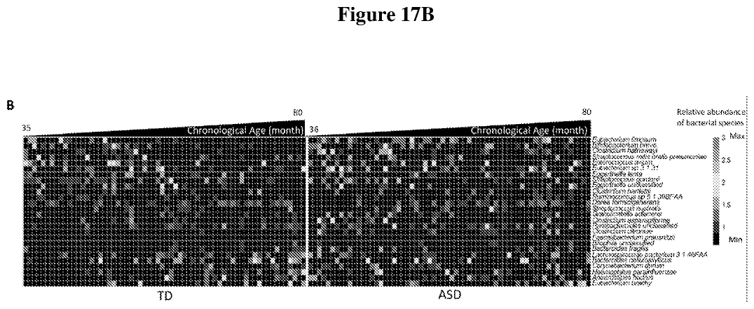

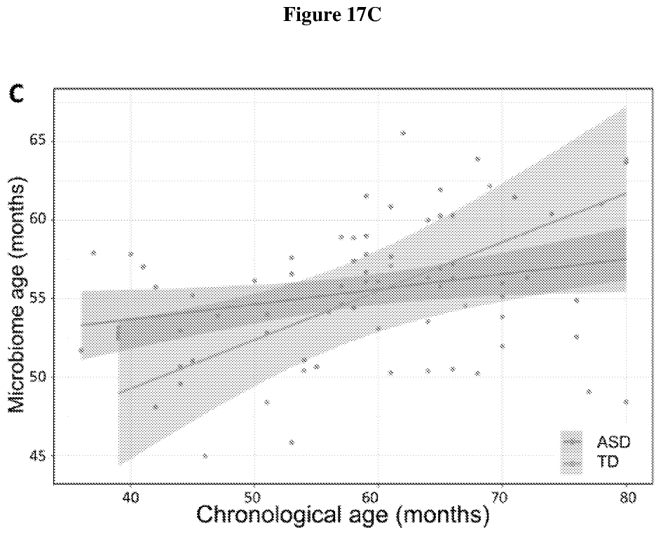

Given the impact of host chronological age on composition and functionality of the gut microbiome, it was hypothesized that age-related bacteria seen in healthy children may develop abnormally in the gut of ASD children. Age-discriminatory taxa were identified in TD children and subsequently investigated their abundance in association with age in ASD children. The relative abundance of fecal bacterial species regressed against the chronologic age of TD children at the time of fecal sample collection, via Random Forest with five times ten-fold cross-validation. Consequently, 26 age-discriminatory bacterial species were discerned, as proxy of “normal” development of children's gut microbiome with age (FIG. 17A and FIG. 17B, left panel). In contrast to the gradual development pattern of the age-discriminatory bacteria taxa with children's chronological age observed in TD children (FIG. 17B, left panel), the abundance and pattern of these age-discriminatory bacteria taxa was substantially disrupted in ASD children, which became age-independent (FIG. 17B, right panel). For instance, the relative abundance of the species Eubacterium limosum and Bifidobacterium breve decreased with age, whereas the species Eubacterium brachy, Haemonphilus parainfluenzae, Bacteroides cellulosilyticus, and Lachnospiraceae bacterium 3_1_46FAA abundance increased with age in TD children (FIG. 17B, left panel). In line with this result, these age-discriminatory bacteria have been associated with healthy growth of children previously (Wong et al., Nutrients 11(8): 1724, 2019). However, the age-associated pattern for these species were lost in children with ASD (FIG. 17B, right panel), suggesting abnormal development of the gut microbiota during early life growth and development in ASD children compared with their age-matched peers.

To validate their finding, the inventors developed a sparse microbiome-age prediction model as a function of the chronological age in TD children based the abundance of the 26 age-discriminatory species (FIG. 17C). In TD children, the predicted microbiome-age grows linearly with children's chronological age, illustrating a steady development landscape of the gut microbiome with age in childhood. However, when employing the microbiome-age model developed in TD children to predict the microbiome-age of ASD children, the inventors discovered that the gut microbiome of ASD children showed under-development in keeping up with the chronological age of the host, as illustrated by the more placid slope observed in the microbiome-age against the chronological-age model of ASD children than that observed in TD children (slope of the linear model: 0.10 versus 0.31 respectively, FIG. 17C). These data altogether indicate that ASD children have impaired development of their gut microbiome during childhood growth, as compared to their peers. Gut microbiome co-evolve with children to develop a mutualistic and symbiotic relationship, abnormal gut microbial development in childhood may have a long-lasting effect in host health.

### REFERENCES

1 Bolger A M, Lohse M, Usadel B. Trimmomatic: a flexible trimmer for Illumina sequence data. Bioinformatics 2014; 30:2114-20.
2 Truong D T, Franzosa E A, Tickle T L, Scholz M, Weingart G, Pasolli E, et al. MetaPhlAn2 for enhanced metagenomic taxonomic profiling. Nat Methods 2015; 12:902-3.
3 Langmead B, Salzberg S L. Fast gapped-read alignment with Bowtie 2. Nat Methods 2012; 9:357-9.
4 Hadley W, Mara A, Jennifer B, Winston C, Lucy M, Romain F, et al. Welcome to the Tidyverse. Journal of Open Source Software 2019; 4:1686.
5 McMurdie P J, Holmes S. phyloseq: an R package for reproducible interactive analysis and graphics of microbiome census data. PLoS One 2013;8:e61217.
6 Segata N, Izard J, Waldron L, Gevers D, Miropolsky L, Garrett W S, et al. Metagenomic biomarker discovery and explanation. Genome Biol 2011;12:R60.
7 Breiman L. Random Forests. Machine Learning 2001; 45:5-32.
8 Cutler D R, Edwards Jr T C, Beard K H, Cutler A, Hess K T, Gibson J, et al. Random forests for classification in ecology. Ecology 2007; 88:2783-92.
9 Robin X, Turck N, Hainard A, Tiberti N, Lisacek F, Sanchez J C, et al. pROC: an open-source package for R and S+ to analyze and compare ROC curves. BMC Bioinformatics 2011; 12:77.

### Example IV: Microbial Markers for Autism

Autism spectrum disorder (ASD) is a complex group of developmental disorders characterized by impaired social interactions and communication together with repetitive behaviors. The purpose of this study is to determine bacterial biomarkers for individuals with autism, as well as to pinpoint probiotic/therapeutic bacteria for autism. The gut bacterial profile is different between autistic children and typically developing children. Gut microbiota is regarded as an important factor in the development of ASD. The practical use of this discovery includes predicting risk for autism in children and microbial transfer and/or supplementation as a potential means to improve behavioral symptom in autistic individuals.

### Methods

### Cohort Description and Study Subjects

A total of 120 Chinese children (aged between 3 and 6 years) were recruited: 61 autism spectrum disorder children and 59 typically developing children. Cohort recruited more male (83%) than female (17%), majority of ASD children were diagnosis with ASD around 3 years old.

The study was approved by The Joint Chinese University of Hong Kong, New Territories East Cluster Clinical Research Ethics Committee (The Joint CUHK-NTEC CREC, CREC Ref. No: 2016.607). All subjects consented to donate fecal samples and to the questionnaire investigation, where written informed consents were obtained. Fecal samples from the study subjects were stored at −80° C. for downstream microbiome analyses.

Families with their children diagnosed with ASD by pediatrician or clinical psychologist according to the standard of the fourth or fifth edition of the Diagnostic and Statistical Manual of Mental Disorders (DSM-IV or DSM-V) will be included. Children without ASD, delays in motor and language development, as well as behaviors as reported by their parents, and those do not have first-degree relatives with ASD will be included as typically developing children. Sixty-five families with children of ASD of Chinese origin and 65 families with typically developing children were divide into 2 groups: case group and control group.

### Fecal DNA Extraction and DNA Sequencing

Fecal bacterial DNA was extracted by Maxwell® RSC PureFood GMO and Authentication Kit (Promega) with modifications to increase the yield of DNA. Approximately 100 mg from each stool sample was pretreated: stool sample suspended in 1 ml ddH₂O and pelleted by centrifugation at 13,000×g for 1 min. Washed sample was added 800ul TE buffer (PH 7.5), 16 ul beta-Mercaptoethanol and 250U lyticase sufficiently mixed and digestion at 37° C. for 90 minutes. Pelleted by centrifugation at 13,000×g for 3 minutes.

After pretreatment, precipitate was resuspended in 800ul CTAB buffer (Maxwell® RSC PureFood GMO and Authentication Kit following manufacturer's instructions) and mixed well. After samples were heated at 95° C. for 5 minutes and cooled down, nucleic acid were released from the samples by vortexing with 0.5 mm and 0.1 mm beads at 2850 rpm for 15 minutes. Following this, 40ul Proteinase K and 20ul RNase A were added and nucleic acid digested at 70° C. for 10 minutes. Finally, supernatant was obtained after centrifugation at 13,000×g, 5 minutes and placed in a Maxwell® RSC instrument for DNA extraction. The extracted fecal DNA was used for ultra-deep metagenomics sequencing via Ilumina Novoseq 6000 (Novogen, Beijing, China).

### Quality Control of Raw Sequences

Raw sequence reads were trimmed by Trimmomatic¹ (Trimmomatic-0.36) firstly and then separation of non-human reads from contaminant host reads. There were some steps to acquire clean reads: 1) Remove adapters; 2) Scan the read with a 4-base wide sliding window, removing reads when the average quality per base drop below 20; 3) Drop reads below the 50 bases long. Trimmed sequence reads were used by KneadData (Reference database: GRCh38 p12) to separate the non-human reads from human reads. Paired-end two reads were concatenated together.

### Analysis of the Bacterial Microbiome

Profiling of the composition of bacterial communities was performed on metagenomic trimmed reads via MetaPhlAn2 (v2.7.5)2. Mapping reads to clade-specific markers gene and annotation of species pangenomes was done through Bowtie2 (v2.3.4.3)3. The output table contained bacterial species and its relative abundance in different levels, from kingdom to strain level.

### Design of Primers and Probes

Primer and probe sequences for the internal control were designed manually on the basis of the conservative fragments in bacterial 16S rRNA genes, and then they were tested using the tool PrimerExpress v3.0 (Applied Biosystems) for determination of Tm, GC content, and possible secondary structures. Degenerate sites were included in the primers and probes to increase target coverage; degenerate sites were not close to 3′ ends of primers and 5′ end of the probes. Amplicon target was nt 1,063-1,193 of the corresponding E. coli genome.

Three bacterial marker candidates identified by previous metagenome sequencing were selected for qPCR quantification, including Alistipes indistinctus (Ai), Anaerotruncus colihominis (Ac) and Eubacterim hallii (Eh). These candidates were identified by AUC value ranking in metagenome study. Primer and probe sequences targeting the gene markers which extracted from MetaPhlAn2 database. Primers were designed using Primer-BLAST in NCBI and probes were designed manually. The primer-probe sets specifically detect the targets and not any other known sequences, as confirmed by Blast search. Each probe carried a 5′reporter dye FAM (6-carboxyfluorescein) or VIC (4,7,20-trichloro-70-phenyl-6-carboxyfluores-cein) and a 3′quencher dye TAMRA (6-carboxytetramethyl-rho-damine). Primers and hydrolysis probes were synthesized by BGI. Nucleotide sequences of the primers and probes are listed below. PCR amplification specificity was confirmed by direct Sanger sequencing of the PCR products.

TABLE 12
Nucleotide sequences of the primers and probes for Ai, Ac and Eh

| | | |
|---|---|---|
| Alistipes | primer F | CGTCTTTACCGGGAGGCAAT |
| indistinctus (Ai) | primer R | AAACCGTCGAAAGGCAGACAGT |
| | probe | TCCGGAGAGCTACCTGATGGCCTGCAAT |
| Anaerotruncus | primer F | TGGCACAGGCTTTTTGGAAT |
| colihominis (Ac) | primer R | CCTGAGACAAGCACCGTTCC |
| | probe | TTACCGGGAACTCAATCCCGCGGAAGATTAT |
| Eubacterium | primer F | AGAGGCACAGCAGCCGAACT |
| hallii (Eh) | primer R | TGTTTGGTCCGTCACCGTCAT |
| | probe | TCGTGCCGCTTACTGGATCTTCCGTAACATT |

### Quantitative PCR

Quantitative PCR (qPCR) amplifications were performed in a 20 uL reaction system of TaqMan Universal Master Mix II (Applied Biosystems) containing 0.3 mmol/L of each primer and 0.2 mmol/L of each probe in MicroAmp fast optical 96-well reaction plates (Applied Biosystems) with adhesive sealing. Thermal cycler parameters of an ABI PRISM 7900HT sequence detection system was 95° C. 10 minutes and (95° C. 15 seconds, 60° C. 1 minute) x45 cycles. A positive/reference control and a negative control (H2O as template) were included within every experiment. Measurements were performed in duplicates for each sample. qPCR data was analyzed using the Sequence DetectionSoftware (Applied Biosystems) with manual settings of threshold=0.05 and baseline from 3-15 cycles for all clinical samples. Experiments were disqualified if their negative control Cq value was<42. Data analysis was carried out according to the ΔCq method, with ΔCq=Cq_{target}−Cq_{control} and relative abundances=POWER (2−ΔCq).

### Results and Findings

### Different Gut Bacterial Profile Between ASD Children and Typically Developing Children

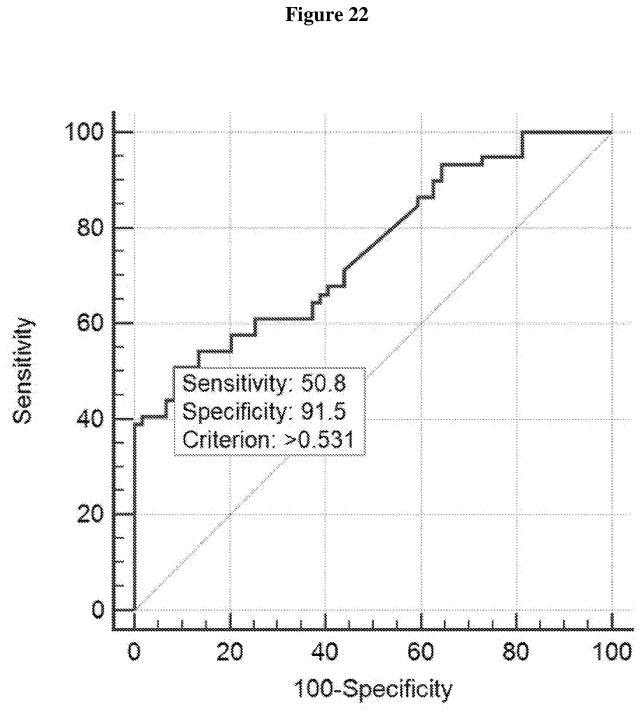

According to the performance of the classification, the species Alistipes indistinctus and Anaerotruncus colihominis (Table 13) showed higher relative abundance in children with ASD than typically developing children. In contrast, the species Eubacterium hallii (Table 14) was depleted in children with ASD as compared to typically developing children. The performance of each marker alone and in combination are shown in Table 15. FIG. 22 shows the ROC curve of combined score. For combined score, it was calculated using a logistic regression model (combined score=I1+β1*Ai+β2*Eh+β3*Ac). In the regression models, I represented the intercepts, R represented the regression coefficients and markers represented the corresponding Cp values.

TABLE 13
Bacterial Species Enriched in Children with Autism Spectrum
Disorder Compared to Typically Developing Children

| Bacterial Species | NCBI: txid |
|---|---|
| Alistipes indistinctus (Ai) | 626932 |
| Anaerotruncus colihominis (Ac) | 169435 |

TABLE 14
Bacterial Species Depleted in Children with Autism Spectrum
Disorder Compared to Typically Developing Children

| Bacterial Species | NCBI: txid |
|---|---|
| Eubacterium hallii (Eh) | 39488 |

TABLE 15
Performance of each marker alone and in combination
with other bacteria in ASD classification

| | AUC | P value | Sensitivity | Specificity |
|---|---|---|---|---|
| Alistipes indistinctus | 0.706 | <0.0001 | 52.46 | 88.14 |
| Eubacterium hallii | 0.626 | 0.0044 | 91.80 | 38.98 |
| Anaerotruncus colihominis | 0.614 | 0.0293 | 42.37 | 83.05 |
| Combined score | 0.754 | <0.0001 | 50.8 | 91.50 |

These bacterial markers can be used separately or in combination to determine the risk of developing ASD in a subject. Standard control value (relative abundance of bacterial species or their combined scores found in typically developing children) can be established to provide a cut-off value to indicate whether or not the subject being examined has an elevated risk for ASD. For both single markers and combined scores, cutoff values are determined by receiver operating characteristic (ROC) analyses that maximized the Youden index (J=Sensitivity+Specificity−1). Pairwise comparison of areas under ROC (AUROCs) for each method/marker was performed using a nonparametric approach.

For example, the cut-off values of Ai, and Ac in this cohort are 0.000000019 and 0.000000758 respectively. The cut-off value of combined score in this cohort is 0.531 (FIG. 22). Subjects having a combined value larger than these cut-off values are deemed to have a higher risk of ASD. The cut-off values of Eh is 0.00129794. Subjects having a value smaller or equal to this cut-off value are deemed to have a higher risk of ASD.

All patents, patent applications, and other publications, including GenBank Accession Numbers and the like, cited in this application are incorporated by reference in the entirety for all purposes.

What is claimed is:

1. A method for treating symptoms of autism spectrum disorder (ASD) in a human child, comprising introducing into the child's gastrointestinal tract an effective amount of one or more of the bacterial species of Faecalibacterium prausnitzi, Roseburia inulinivorans, Eubacterium hallii, Dorea longicatena, or Eubacterium siraeum.

2. The method of claim 1, wherein the introducing step comprises oral administration to the subject a composition comprising an effective amount of the one or more of the bacterial species.

3. The method of claim 1, wherein the introducing step comprises delivery to the small intestine, ileum, or large intestine of the subject a composition comprising an effective amount of the one or more of the bacterial species.

4. The method of claim 1, wherein the introducing step comprises fecal microbiota transplantation (FMT).

5. The method of claim 4, wherein the FMT comprises administration to the child a composition comprising processed donor fecal material.

6. The method of claim 2, wherein the composition is orally administered.

7. The method of claim 2, wherein the composition is directly deposited to the child's gastrointestinal tract.

8. The method of claim 1, wherein the level or relative abundance of the one or more of the bacterial species is determined in a first stool sample obtained from the child prior to the introducing step and in a second stool sample obtained from the child after the introducing step.

9. The method of claim 8, wherein the level of the one or more of the bacterial species is determined by quantitative polymerase chain reaction (PCR).

10. A method for treating symptoms of autism spectrum disorder (ASD) in a human child, comprising reducing the level or relative abundance of one or more of the bacterial species of Clostridium nexile, Dialister invisus, Clostridium bolteae, Clostridium symbiosum, Eubacterium limosum, Clostridiales bacterium_1_7_47FAA, Clostridium ramosum, Anaerotruncus colihominis, Clostridium citroniae, or Alistipes indistinctus in the child's gastrointestinal tract.

11. The method of claim 10, wherein the reducing step comprising FMT.

12. The method of claim 10, wherein the reducing step comprises treating the subject with an anti-bacterial agent.

13. The method of claim 12, wherein a composition comprising processed donor fecal material is introduced to the gastrointestinal tract of the subject after the subject is treated with the anti-bacterial agent.

14. The method of claim 13, wherein the composition is orally administered.

15. The method of claim 13, wherein the composition is directly deposited to the gastrointestinal tract of the child.

16. The method of claim 10, wherein the level or relative abundance of the one or more of the bacterial species is determined in a first stool sample obtained from the child prior to the reducing step and in a second stool sample obtained from the child after the reducing step.

17. The method of claim 16, wherein the level of the one or more bacterial species is determined by quantitative polymerase chain reaction (PCR).

18. A kit for treating symptoms of ASD, comprising: a first container containing a first composition comprising (i) an effective amount of one of the bacterial species set forth in Table 1, or (ii) an effective amount of an anti-bacterial agent that suppresses growth of one of the bacterial species set forth in Table 2, and a second container containing a second composition comprising (i) an effective amount of another one of the bacterial species set forth in Table 1, or (ii) an effective amount of an anti-bacterial agent that suppresses growth of another one of the bacterial species set forth in Table 2.

19. The kit of claim 18, wherein the first composition comprises processed donor fecal material for FMT.

20. The kit of claim 18 or 19, wherein the first composition is formulated for oral administration.

21. The kit of claim 18, wherein the second composition is formulated for oral administration.

22. The kit of claim 19, wherein both the first and second compositions are formulated for oral ingestion.

23. A method for determining risk for autism spectrum disorder (ASD) in a human child, comprising:
(1) determining, in a stool sample from the child, the relative abundance of any one of the bacterial species set forth in Table 1 or 2; and
(2) detecting the relative abundance from step (1) being no lower than the cutoff value in Table 1 or a standard control value or being lower than the cutoff value or a standard control value in Table 2 and determining the child as not having increased risk for ASD; or detecting the relative abundance from step (1) being lower than the cutoff value in Table 1 or a standard control value or being no lower than the cutoff value in Table 2 or a standard control value and determining the child as having an increased risk for ASD.

24. A method for assessing risk for autism spectrum disorder (ASD) in two human children, comprising:
(1) determining, in a stool sample from each of the two children, the relative abundance of any one of the bacterial species set forth in Table 1 or 2;
(2) determining the relative abundance of a bacterial species set forth in Table 1 from step (1) being higher in the stool sample from the first child or the relative abundance of a bacterial species set forth in Table 2 from step (1) being lower in the stool sample from the first child; and
(3) determining the second child as having a higher risk for ASD than the first child.

25. A method for determining risk for autism spectrum disorder (ASD) in a human child, comprising:
(1) obtaining in a stool sample from the child a value of (a) the relative abundance of Alistipes indistinctus (Ai) or Anaerotruncus colihominis (Ac), or (b) the combined score of levels of three bacterial species Ai, Ac, and Eubacterium hallii (Eh), which is calculated by by I1+β1*Ai+β2*Eh+β3*Ac; and
(2) detecting the value to be higher than a standard control value and determining the individual as having increased risk of ASD.

26. A method for determining risk for autism spectrum disorder (ASD) in a human child, comprising:
(1) obtaining in a stool sample from the child a value of the relative abundance of Eubacterium hallii (Eh); and
(2) detecting the value to be lower than a standard control value and determining the individual as having increased risk of ASD.

27. The method of any one of claims 23-26, wherein the relative abundance of the bacterial species is determined by quantitative PCR.

28. A method for assessing risk for autism spectrum disorder (ASD) in a human child, comprising:
(1) determining, in a stool sample from the child, the level or relative abundance of one or more of the bacterial species set forth in Table 3;
(2) determining the level or relative abundance of the same bacterial species in a stool sample from a reference cohort comprising normal and ASD children;
(3) generating decision trees by random forest model using data obtained from step (2) and running the level or relative abundance of one or more of the bacterial species from step (1) down the decision trees to generate a risk score; and
(4) determining the child with a risk score greater than 0.5 as having an increased risk for ASD and determining the child with a risk score no greater than 0.5 as having no increased risk for ASD.

29. The method of claim 28, wherein the one or more bacterial species comprise Alistipes indistinctus.

30. The method of claim 28, wherein the one or more bacterial species comprise Alistipes indistinctus, candidate division TM7 single-cell isolate TM7c, and Streptococcus cristatus.

31. The method of claim 28, wherein the one or more bacterial species comprise Alistipes indistinctus, candidate division TM7 single-cell isolate TM7c, Streptococcus cristatus, Eubacterium_limosum, and Streptococcus_oligofermentans.

32. A kit for assessing risk for autism spectrum disorder (ASD), comprising reagents for detecting one or more of the bacterial species set forth in Table 1, 2, or 3.

33. The kit of claim 32, wherein the reagents comprise a set of oligonucleotide primers for amplification of a polynucleotide sequence from any one of the bacterial species set forth in Table 1, 2, or 3.

34. The kit of claim 33, wherein the amplification is PCR, preferably quantitative PCR.

35. A method for determining developmental age of a child, comprising the steps of:
(a) quantitatively determining the relative abundance of one or more bacterial species selected from Table 8 or 9 in a stool sample taken from the child;
(b) quantitatively determining the relative abundance of the one or more bacterial species in a stool sample taken from a reference cohort consisting of typically developing children;
(c) generating decision trees by random forest model using data obtained from step (b); and
(d) running the relative abundances obtained from step (a) down the decision trees from step (b) to generate a developmental age for the child.

36. The method of claim 35, wherein the one or more bacterial species comprise Streptococcus gordonii, Enterococcus avium, Eubacterium_sp_3_1_31, Clostridium hathewayi, and Corynebacterium durum.

37. The method of claim 35, wherein the one or more bacterial species comprise Streptococcus gordonii, Enterococcus avium, Eubacterium_sp_3_1_31, and Clostridium hatheway.

38. The method of claim 35, wherein the one or more bacterial species comprise Streptococcus gordonii, Enterococcus avium, and Eubacterium_sp_3_1_31.

39. The method of claim 35, wherein the one or more bacterial species comprise Streptococcus gordonii and Enterococcus avium.

40. The method of claim 35, wherein the one or more bacterial species comprise Streptococcus gordonii.

41. The method of claim 35, wherein the child is between about 3 to about 6 years old.

42. A kit for determining developmental age of a child, comprising: a first container containing a first reagent for detecting a first bacterial species set forth in Table 8 or 9, and a second container containing a second reagent for detecting a second bacterial species set forth in Table 8 or 9.

43. The kit of claim 42, comprising three or more containers each of which containing a reagent for detecting a different bacterial species set forth in Table 8 or 9.

44. The kit of claim 42, comprising two or more containers each of which containing a reagent for detecting a different bacterial species selected from the group consisting of (1) Streptococcus gordonii, Enterococcus avium, Eubacterium_sp_3_1_31, Clostridium hathewayi, and Corynebacterium durum; (2) Streptococcus gordonii, Enterococcus avium, Eubacterium_sp_3_1_31, and Clostridium hatheway; (3) Streptococcus gordonii, Enterococcus avium, and Eubacterium_sp_3_1_31; or (4) Streptococcus gordonii and Enterococcus avium.

45. The kit of claim 42, wherein the reagents comprise a set of oligonucleotide primers for amplification of a polynucleotide sequence from any one of the bacterial species set forth in Table 8 or 9.

46. The kit of claim 45, wherein the amplification is PCR.

47. The kit of claim 46, wherein the PCR is quantitative PCR (qPCR).

48. A method for promoting growth and development of a child, comprising administering to the child an effective amount of one or more bacterial species selected from Table 8.

49. The method of claim 48, wherein the child is between about 3 to about 6 years old.

50. A kit for promoting growth and development of a child, comprising: a first container containing a first composition comprising (i) an effective amount of one of the bacterial species set forth in Table 8, and a second container containing a second composition comprising (i) an effective amount of another one of the bacterial species set forth in Table 8.

51. The kit of claim 50, wherein the first or second composition comprises processed donor fecal material for FMT.

52. The kit of claim 50 or 51, wherein the first composition is formulated for oral administration.

53. The kit of claim 50 or 51, wherein the second composition is formulated for oral administration.

54. The kit of claim 51, wherein both the first and second compositions are formulated for oral ingestion.
